SEQUENCE LISTING (30 sequences)

SEQ ID NO: 1 - protein, 134 aa, Mus musculus
DVQLQESGPGLVNPSQSLSLTCTVTGYSITGDYAWNWIRQFPGNKLEWMGYISYSGLTSYNPSLKSRISITRDTSKNQFFLQLNSVATEDTATYYCTSFYYGYWYFDVWGAGTTVTVSSAKTTPPSVYPLKGEF

SEQ ID NO: 2 - protein, 123 aa, Mus musculus
DIQMTQSPASLSASVGETVTITCRASENIYSFLTWYLQKQGKSPQLLVNNAKTLAEGVPSRFSGSGSGSQFSLKINSLQPEDFGTYYCQHHYGTPYTFGGGTKLEIKRADAAPTVSIFPPSSK

SEQ ID NO: 3 - protein, 92 aa, Mus musculus
RQFPGNKLEWMGYINYSGITSYNPSLKSRFSITRDTSKNQFFLQLNSVTTEDTATYYCASWYYGNWYFDVWGAGTTVTVSSAKTTPPSVYPL

SEQ ID NO: 4 - protein, 126 aa, Mus musculus
DIQMTQSPASLSVSVGETVTITCRASENIYSNLAWYQQKQGNSPQLLVYAATNLADGVPSRFSGSGSGTQYSLKINSLQSEDFGNYYCQHFWGTPWTFGGGTKLEIKRADAAPTVSIFPPSSKGEF

SEQ ID NO: 5 - protein, 94 aa, Mus musculus
VRQPPGKGLEWLGVLWAGGSTNYNSALMSRLSIHKDNSKSQFFLKMNSLQADDTAMYYCARARSAMDYWGQGTSVTVSSAKTTPPSVYPLKGEF

SEQ ID NO: 6 - protein, 122 aa, Mus musculus
DIVLTQSPASLAVSLGQRATISCKASQSVDYDDNSYMNWYQQKPGQPPKLLIYAASNLESGIPARFSGSGSGTDFTLNIHPVEEEDAATYYCQQSNEDPYTFGGGTKLEIKRADAAPTVSIF

SEQ ID NO: 7 - protein, 57 aa, Mus musculus
RQFPGNKLEWMGYINYSGITSYNPSLKSRFSITRDTSKNQFFLQLNSVTTEDTATYY

SEQ ID NO: 8 - protein, 14 aa, Mus musculus
CASWYYGNWYFDVW

SEQ ID NO: 9 - protein, 8 aa, Mus musculus
GAGTTVTV

SEQ ID NO: 10 - protein, 13 aa, Mus musculus
SSAKTTPPSVYPL

SEQ ID NO: 11 - protein, 87 aa, Mus musculus
DIQMTQSPASLSVSVGETVTITCRASENIYSNLAWYQQKQGNSPQLLVYAATNLADGVPSRFSGSGSGTQYSLKINSLQSEDFGNYY

SEQ ID NO: 12 - protein, 11 aa, Mus musculus
CQHFWGTPWTF

SEQ ID NO: 13 - protein, 10 aa, Mus musculus
GGGTKLEIKR

SEQ ID NO: 14 - protein, 18 aa, Mus musculus
ADAAPTVSIFPPSSKGEF

SEQ ID NO: 15 - protein, 58 aa, Mus musculus
VRQPPGKGLEWLGVLWAGGSTNYNSALMSRLSIHKDNSKSQFFLKMNSLQADDTAMYY

SEQ ID NO: 16 - protein, 11 aa, Mus musculus
CARARSAMDYW

SEQ ID NO: 17 - protein, 8 aa, Mus musculus
GQGTSVTV

SEQ ID NO: 18 - protein, 17 aa, Mus musculus
SSAKTTPPSVYPLKGEF

SEQ ID NO: 19 - protein, 91 aa, Mus musculus
DIVLTQSPASLAVSLGQRATISCKASQSVDYDDNSYMNWYQQKPGQPPKLLIYAASNLESGIPARFSGSGSGTDFTLNIHPVEEEDAATYY

SEQ ID NO: 20 - protein, 7 aa, Mus musculus
CQQSNED

SEQ ID NO: 21 - protein, 14 aa, Mus musculus
PYTFGGGTKLEIKR

SEQ ID NO: 22 - protein, 10 aa, Mus musculus
ADAAPTVSIF

SEQ ID NO: 23 - protein, 95 aa, Mus musculus
DVQLQESGPGLVNPSQSLSLTCTVTGYSITGDYAWNWIRQFPGNKLEWMGYISYSGLTSYNPSLKSRISITRDTSKNQFFLQLNSVATEDTATYY

SEQ ID NO: 24 - protein, 12 aa, Mus musculus
CTSFYYGYWYFD

SEQ ID NO: 25 - protein, 10 aa, Mus musculus
VWGAGTTVTV

SEQ ID NO: 26 - protein, 17 aa, Mus musculus
SSAKTTPPSVYPLKGEF

SEQ ID NO: 27 - protein, 87 aa, Mus musculus
DIQMTQSPASLSASVGETVTITCRASENIYSFLTWYLQKQGKSPQLLVNNAKTLAEGVPSRFSGSGSGSQFSLKINSLQPEDFGTYY

SEQ ID NO: 28 - protein, 11 aa, Mus musculus
CQHHYGTPYTF

SEQ ID NO: 29 - protein, 12 aa, Mus musculus
GGGTKLEIKRAD

SEQ ID NO: 30 - protein, 13 aa, Mus musculus
AAPTVSIFPPSSK

(19) United States
(12) Patent Application Publication
(10) Pub. No.: US 2011/0229908 A1
(43) Pub. Date: Sep. 22, 2011

(54) METHODS AND PRODUCTS FOR MEASURING FREE IMMUNOGLOBULIN LIGHT CHAIN MOLECULES

(76) Inventors: Royston Jefferis, Birmingham (GB); Mark Cobbold, Birmingham (GB); Timothy Plant, Solihull (GB); Delia Margaret Goodall, Solihull (GB); Mark Trehane Drayson, Cornwall (GB)

(21) Appl. No.: 13/125,283

(22) PCT Filed: Oct. 22, 2009
(86) PCT No.: PCT/GB2009/002523

§ 371 Date: Jun. 7, 2011

(30) Foreign Application Priority Data
Oct. 28, 2008 (GB) 0819720.4

(51) Int. Cl. G01N 33/53; C07K 16/00; C12N 5/16

(52) U.S. Cl. 435/7.1; 436/501; 530/388.1; 435/326

(57) ABSTRACT

The present invention provides improved methods for measuring the ratio of free K immunoglobulin light chain molecules to free λ immunoglobulin light chain molecules in a test sample, using a monoclonal antibody that specifically binds to free K and a monoclonal antibody that specifically binds to free λ. The application also provides specific monoclonal antibodies that can be used in the method of the invention, as well as kits containing said monoclonal antibodies.

The present invention relates to improved methods of detecting the presence of serum immunoglobulins, and reagents to be used in such methods.

The measurement of serum immunoglobulins have much clinical application as part of clinical practice in the investigation of patients with dysregulated immunoglobulin secretion including plasma cell dyscrasias, immunoglobulin deposition diseases and immunodeficiency. In addition, they are commonly measured in patients to monitor response of plasma cell dyscrasias and other B cell malignancies to cytotoxic therapy. In the last 5 years the measurement of free light chain immunoglobulin fragments in serum has been shown to be of clinical value in the screening of patients with suspected myeloma, plasmacytoma, light chain amyloid, Waldenstrom's, B-cell chronic lymphocytic leukaemia and monoclonal gammopathy of undetermined significance (MGUS).

Monoclonal gammopathy is a disorder caused by abnormal proliferation of a single clone of plasma cells. Monoclonal gammopathies may be present in a wide spectrum of diseases, that include malignancies of plasma cells or B lymphocytes (multiple myeloma (MM), macroglobulinaemia, plasmacytoma, B-cell lymphoma), disorders involving monoclonal proteins of abnormal structure (primary systemic amyloidosis (AL), light chain deposition disease (LCDD), cryoglobulinaemia), and apparently benign, premalignant conditions (monoclonal gammopathy of undetermined significance (MGUS), asymptomatic MM). While the specific clinical diagnosis is dependent on a number of clinical features, as well as other laboratory assessments, the presence of the monoclonal gammopathy is a laboratory diagnosis.

Plasma cells secrete immunoglobulins and each clone of plasma cells produces a single type of immunoglobulin. In monoclonal gammopathies, there is an expansion of a single plasma cell clone, and consequently an increase in the monoclonal immunoglobulin produced by this clone. The presence of a monoclonal immunoglobulin (M-protein) serves as a marker of the clonal proliferation of the plasma cells, and is diagnostic for a monoclonal gammopathy. In addition, the disease course can be monitored by following the concentration of the monoclonal immunoglobulin.

Immunoglobulin molecules are made up of two identical heavy and two identical light chain polypeptide molecules. There are five types of heavy chains: gamma, alpha, mu, delta, and epsilon; and two types of light chains: kappa (κ) and lambda (λ). Both the heavy and light chain molecules are produced within the plasma cell and then assembled into an immunoglobulin molecule, forming the 5 classes of immunoglobulins: (Ig)—IgG, IgA, IgM, IgD, and IgE, each of which is constructed with identical either κ or λ light chain molecules. Any of these 5 classes may be associated with monoclonal gammopathies. Protein electrophoresis and immunofixation electrophoresis (both serum and urine) are traditionally usually used to identify monoclonal immunoglobulin molecules (M-proteins).

Normal and abnormal plasma cells produce more light chains than heavy chains, and these excess light chains are released into the bloodstream. These unbound, or free, light chains (FLCs) are typically cleared rapidly and metabolized by the kidneys. Increases in serum free light chains may occur due to decreased renal clearance, increased polyclonal immunoglobulin production, or monoclonal gammopathies. For patients with impaired renal clearance or polyclonal increases in immunoglobulins (hypergammaglobulinaemia—seen in liver disease, autoimmune diseases, or infectious processes), serum free light chain levels may be elevated, but the free light chain κ:λ ratio will be normal. For patients with an abnormal expansion of either a kappa- or lambda-producing plasma cell clone, the serum free light chain κ:λ ratio usually becomes abnormal. This is because the excess production of one light chain type from the abnormally expanded plasma cell clone perturbs the normal light chain ratio generated by polyclonal light chain production from twice as many kappa plasma cells as lambda plasma cells. This ratio is a very sensitive diagnostic test for abnormally expanded plasma cell clones.

Protein electrophoresis and immunofixation electrophoresis of serum and 24-hour urine are the standard assays to detect both whole immunoglobulin M-proteins and free light chain M-proteins (Bence Jones Proteins). Protein electrophoresis is used both to identify the presence of a monoclonal immunoglobulin spike and to quantitate the concentration of the M-protein by densitometry. Immunofixation electrophoresis is performed to characterize the type of monoclonal protein (γ, α, μ, δ or ε heavy chain; κ or λ light chain). Immunofixation electrophoresis is more sensitive than protein electrophoresis for detecting low levels of M-protein that may be present in diseases such as light chain amyloid, oligosecretory myeloma, and plasmacytomas. However, immunofixation is unable to quantitate the paraprotein present.

Free light chain M-proteins are secreted by plasma cells, filtered at the renal glomerulus and reabsorbed by the renal tubules and so only appear in the urine when the renal threshold for reabsorption is exceeded. Accordingly FLC are not normally detectable in urine. When a malignant plasma cell clone arises, secreting FLC M-protein then even at low levels of secretion the serum FLC κ:λ ratio will be perturbed beyond the normal range. In contrast the malignant clone must grow considerably more before its FLC M-protein secretion will exceed renal tubular capacity for FLC reabsorption allowing the M-protein FLC to become detectable in urine by immunofixation. In analogy with measurement of glucose in diabetes it would be of much better clinical utility to measure FLC in serum not urine.

There are existing assays available that allow for the measurement of free light chains within serum. However, there are a number of disadvantages associated with the existing assays. The available assays rely on the production of polyclonal antiserum produced in sheep to measure FLC levels, and it is well known that polyclonal antiserum is subject to batch to batch variation. This includes some batches having very poor reactivity and/or specificity. Existing assays do not enable the determination of both κ and λ free light chain types simultaneously, therefore to detect both FLC forms, two kits must be used in parallel which not only increases the likelihood of errors being made in performing the assay, but is also time consuming and wasteful of laboratory resources. The existing assays are prone to the phenomenon of antigen excess and so in some patient's sera the assay fails to detect grossly elevated levels of FLC. The existing assays can only measure FLC levels accurately over a limited range of concentrations. In disease serum FLC levels may be ten fold lower than normal or a thousand fold higher than normal and in these samples the existing assay has to be repeated as many as four times at successively greater dilutions to attain an accurate measurement of FLC levels. Available FLC assays have not been shown to be reliable to detect light chains in urine thus electrophoresis and densitometry or an immunoprecipitation methodology is employed.

The inventors have devised an improved method for measuring the ratio of free κ immunoglobulin light chain molecules to free λ immunoglobulin light chain molecules in a test sample that overcomes the problems set out above. The method is based on the development of monoclonal antibodies which specifically bind to free κ or free λ immunoglobulin light chain molecules. The monoclonal antibodies used in the improved method have much improved consistency and sensitivity over the polyclonal antibodies used in the existing assays, and do not rely on an animal system for production.

Furthermore the improved method of the invention can detect both κ and λ free light chain types simultaneously; the method can measure light chains in urine and serum equally effectively; and it provides a four decade range of sensitivity and is much faster and less wasteful than the existing assay. These advantages give the method of the invention significant improvements over existing methods of measuring the ratio of free κ to λ immunoglobulin light chain molecules in a test sample.

A first aspect of the invention provides a method of measuring the ratio of free κ immunoglobulin light chain molecules to free λ immunoglobulin light chain molecules in a test sample comprising:
(i) measuring the amount of free κ in the test sample using a monoclonal antibody, or an antigen binding fragment or variant of said monoclonal antibody, that specifically binds to free κ;
(ii) measuring the amount of free λ in the test sample using a monoclonal antibody, or an antigen binding fragment or variant of said monoclonal antibody, that specifically binds to free λ;
(iii) calculating the ratio of free κ to free λ levels in the test sample.

The method of the first aspect of the invention can be used to determine the ratio of free κ to free λ immunoglobulin light chain molecules in a test sample. It therefore has much application for determining whether a subject has, or is likely to develop, a malignant plasma cell disease. Also the method can be used to measure response of these diseases to therapy and to monitor for subsequent relapse of these diseases. Examples of such diseases are set out in the discussion above and further below in relation to another aspect of the invention.

A detailed protocol for the performance of one embodiment of this method of the invention is provided in the accompanying example below.

At the time of developing the invention, it was well known in the field that κ and λ immunoglobulin light chain molecules are inherently highly variable in amino acid sequence. This variability is present as there are around 37 different functional genes encoding κ and around 30 different functional genes encoding λ polypeptide. Furthermore, this variability is increased due to the presence of somatic mutations in the encoding genes, and also the diversity of the J regions which comprise these molecules. As will be appreciated, variability in amino acid sequence can alter the structure of epitopes in the polypeptide recognised by antibodies.

This variability has lead to a prejudice against the development of assays using monoclonal antibodies to routinely measure the ratio of free κ to free λ immunoglobulin light chain molecules in a test sample from a wide range of different subjects. This is because, where there is a malignant plasma cell disease, the free light chain κ or λ molecules will be themselves monoclonal, as they are derived from a malignant plasma cell and thus will have identical amino acid sequence and structure unique to each patient. A monoclonal antibody (mAb) typically only detects a single epitope or antigenic structure on the free κ or free λ molecule. Hence to be of use in such an assay, the monoclonal antibody must recognise and bind to an epitope displayed on substantially all κ or free λ molecules, regardless of any variability.

It was viewed in the field of the invention as being highly unlikely that any monoclonal antibodies could be developed that would specifically recognise all of these different free κ or free λ molecules. It is for this reason that the existing assays rely on the use of polyclonal antiserum to free κ and free λ immunoglobulin light chain molecules, since this allows the assays to recognise a wide range of different free κ or free λ immunoglobulin light chain molecules. However, as stated above, there are a large number of disadvantages with the presently available assays.

After screening many different monoclonal antibodies, the inventors surprisingly identified specific monoclonal antibodies that can be used effectively to measure the ratio of free κ to free λ immunoglobulin light chain molecules in test samples from a hundred normal individuals, four hundred Myeloma Trial Patients and over a thousand individuals with plasma cell dyscrasias. This finding is surprising given the highly variable in amino acid sequence of κ and λ immunoglobulin light chain molecules.

Three monoclonal antibodies were identified that can specifically bind to a wide range of different free κ immunoglobulin light chain molecules, but not to κ light chain molecules bound into whole immunoglobulin or to λ light chain molecules. These have been termed KA1, 1B6 and B3B4.

The antibody produced by the hybridoma cell line KA1 as deposited under Budapest Treaty at the ECACC ((European Collection of Cell Cultures) CEPR (Centre for Emergency Preparedness and Response) Porton Down) under reference number 08071702 is a particularly preferred example of a monoclonal antibody that can bind to free κ.

The antibody produced by the hybridoma cell line 1B6 as deposited under Budapest Treaty at ECACC (details above) under reference number 08071704 is also a particularly preferred example of a monoclonal antibody that can bind to free κ.

The antibody produced by the hybridoma cell line B3B4 as deposited under Budapest Treaty at ECACC (details above) under reference number 08071705 is also a particularly preferred example of a monoclonal antibody that can bind to free κ.

Two monoclonal antibodies were identified that can specifically bind to a wide range of different free λ immunoglobulin light chain molecules, but not to λ light chain molecules bound into whole immunoglobulin or to κ light chain molecules. These have been termed 21C3 and 21E9.

The antibody produced by the hybridoma cell line 21C3 as deposited under Budapest Treaty at ECACC (details above) under reference number 08071701 is a particularly preferred example of a monoclonal antibody that can bind to free λ.

The antibody produced by the hybridoma cell line 21 E9 as deposited under Budapest Treaty at ECACC (details above) under reference number 08071703 is a particularly preferred example of a monoclonal antibody that can bind to free λ.

It can be seen from the accompanying examples that the monoclonal antibodies bind specifically to their antigens with a high sensitivity. Assays using such molecules perform well for both urine and serum samples, with high sensitivity and specificity.

The development of such monoclonal antibodies has allowed the inventors to develop the method of the first aspect of the invention.

The method of the invention can also use further monoclonal antibodies that specifically bind to free κ molecule or free λ molecule.

By “specifically binds” we include that the monoclonal antibody used in this aspect of the invention do not significantly bind to any other molecule than the antigen. Hence the monoclonal antibody that specifically binds to free κ molecule does not bind to any significant degree other antigen; and preferably does not bind at all to kappa light chain molecules bound into whole immunoglobulin or to lambda light chain molecules. Similarly, monoclonal antibody that specifically binds to free λ molecule does not bind to any significant degree to any other antigen; and preferably does not bind at all to bound lambda or to kappa light chain molecules.

It is preferred that the monoclonal antibody specifically binds to a wide range of different free κ or λ immunoglobulin light chain molecules. In the present case the inventors screened antibodies derived from over 20 hybridoma clones against many hundreds of clinical samples to identify those antibodies with optimal assay performance. To be useful in the method of the invention, this subset of antibodies is further reduced by selecting only those antibodies with optimal signal to noise characteristics.

As is well known in the field, a monoclonal antibody consists of four polypeptides—two heavy chains and two light chains joined to form a “Y” shaped molecule with identical binding sites at each of the tips of the two arms of the Y. The amino acid sequence in the tips of the “Y” varies greatly among different antibodies. This variable region, composed of 110-130 amino acids, give the antibody its specificity for binding antigen. The variable region includes the ends of the light and heavy chains. The constant region determines the mechanism used to destroy antigen.

The variable region contains framework regions (FR) which form a beta-sheet structure which serves as a scaffold which has a barrel like structure (sometimes terms a beta-barrel). The tips of this “barrel” have amino acid sequences which are unique to each antibody and bind to the antigen which the antibody is specific for. There are three such regions and because their unique sequence determines what an antibody is specific for, these are termed complementarity determining regions, or CDRs (CDR1, CDR2 and CDR3). CDR3 exhibits the most variability and the sequence of this region is unique to each antibody.

Using routine techniques of recombinant DNA technology, it is possible to produce other antibodies or binding molecules which retain the specificity of the original monoclonal antibody. Such techniques may involve introducing DNA encoding the immunoglobulin variable region, or the complementarity determining regions (CDRs), of an antibody to the constant regions, or constant regions plus framework regions, of a different immunoglobulin.

Against this background, the inventors then obtained the amino acid sequence of the variable regions of monoclonal antibodies that can be used in the method of the invention.

An embodiment of the first aspect of the invention is wherein the variable region of the heavy chain of the monoclonal antibody that specifically binds to free κ comprises the amino acid sequence:

(SEQ ID NO: 1)
DVQLQESGPGLVNPSQSLSLTCTVTGYSITGDYAWNWIRQFPGNKLEWM
GYISYSGLTSYNPSLKSRISITRDTSKNQFFLQLNSVATEDTATYYCTS
FYYGYWYFDVWGAGTTVTVSSAKTTPPSVYPLKGEF.

This amino acid sequence corresponds to the variable region of the heavy chain of a monoclonal antibody designated KA1.

A further embodiment of the first aspect of the invention is wherein the variable region of the light (kappa) chain of the monoclonal antibody that specifically binds to free κ comprises the amino acid sequence:

(SEQ ID NO: 2)
DIQMTQSPASLSASVGETVTITCRASENIYSFLTWYLQKQGKSPQLLVN
NAKTLAEGVPSRFSGSGSGSQFSLKINSLQPEDFGTYYCQHHYGTPYTF
GGGTKLEIKRADAAPTVSIFPPSSK

This amino acid sequence corresponds to the variable region of the light chain of a monoclonal antibody designated KA1.

Preferably the monoclonal antibody that specifically binds to free κ comprises the amino acid sequence of SEQ ID NO:1 and SEQ ID NO:2.

Preferably the monoclonal antibody that specifically binds to free κ is produced by the hybridoma cell line KA1, deposited under Budapest Treaty at the ECACC under reference number 08071702.

An embodiment of the first aspect of the invention is wherein the variable region of the heavy chain of the monoclonal antibody that specifically binds to free κ comprises the amino acid sequence:

(SEQ ID NO: 3)
RQFPGNKLEWMGYINYSGITSYNPSLKSRFSITRDTSKNQFFLQLNSVT
TEDTATYY CASWYYGNWYFDVWGAGTTVTVSSAKTTPPSVYPL

This amino acid sequence corresponds to the variable region of the heavy chain of a monoclonal antibody designated 1B6.

A further embodiment of the first aspect of the invention is wherein the variable region of the light (kappa) chain of the monoclonal antibody that specifically binds to free κ comprises the amino acid sequence:

(SEQ ID NO: 4)
DIQMTQSPASLSVSVGETVTITCRASENIYSNLAWYQQKQGNSPQLLVY
AATNLADGVPSRFSGSGSGTQYSLKINSLQSEDFGNYYCQHFWGTPWTF
GGGTKLEIKRADAAPTVSIFPPSSKGEF.

This amino acid sequence corresponds to the variable region of the light chain of a monoclonal antibody designated 1B6.

Preferably the monoclonal antibody that specifically binds to free κ comprises the amino acid sequence of SEQ ID NO:3 and SEQ ID NO:4.

Preferably the monoclonal antibody that specifically binds to free κ is produced by the hybridoma cell line 1B6, deposited under Budapest Treaty at the ECACC under reference number 08071704.

A further preferred monoclonal antibody that specifically binds to free κ is produced by the hybridoma cell line B3B4, deposited under Budapest Treaty at the ECACC under reference number 08071705.

An embodiment of the first aspect of the invention is wherein the variable region of the heavy chain of the monoclonal antibody that specifically binds to free λ comprises the amino acid sequence:

(SEQ ID NO: 5)
VRQPPGKGLEWLGVLWAGGSTNYNSALMSRLSIHKDNSKSQFFLKMNS
LQADDTAMYYCARARSAMDYWGQGTSVTVSSAKTTPPSVYPLKGEF

This amino acid sequence corresponds to the variable region of the heavy chain of a monoclonal antibody designated 21C3.

A further embodiment of the first aspect of the invention is wherein the variable region of the light (kappa) chain of the monoclonal antibody that specifically binds to free λ comprises the amino acid sequence:

(SEQ ID NO: 6)
DIVLTQSPASLAVSLGQRATISCKASQSVDYDDNSYMNWYQQKPGQPPK
LLIYAASNLESGIPARFSGSGSGTDFTLNIHPVEEEDAATYYCQQSNED
PYTFGGGTKLEIKRADAAPTVSIF

This amino acid sequence corresponds to the variable region of the light chain of a monoclonal antibody designated 21C3.

Preferably the monoclonal antibody that specifically binds to free λ comprises the amino acid sequence of SEQ ID NO:5 and SEQ ID NO:6.

Preferably the monoclonal antibody that specifically binds to free λ is produced by the hybridoma cell line 21C3, deposited under Budapest Treaty at the ECACC under reference number 08071701.

A further preferred monoclonal antibody that specifically binds to free λ is produced by the hybridoma cell line 21E9, deposited under Budapest Treaty at the ECACC under reference number 08071703.

As used in the present disclosure the term “antibody” should, unless the context requires otherwise, also be taken to encompass antigen-binding fragments of antibodies, as well as antigen-binding variant of such antibodies or antibody fragments. Such fragments and variants can be used as monoclonal antibodies in the method of this aspect of the invention.

By “antigen binding fragment or variant thereof” we therefore include those molecules that retain the binding specificity of the monoclonal antibody to the antigen; in this case, either free κ immunoglobulin light chain molecules or free λ immunoglobulin light chain molecules. Methods of determining whether a fragment or variant retains said antigen binding specificity are routine in the art and would be well known to the skilled person; for example routine immunological assays.

Any suitable antigen-binding fragment(s) or variants of the antibodies may be prepared according to techniques known to those skilled in the art. The smallest functional antigen-binding fragments or variants of antibodies may comprise the variable regions of either the heavy (VH) or light (VL) chains of such antibodies. These fragments may have a molecular weight of approximately 13 kDa, or less than one-tenth the typical size of a full antibody.

Antigen-binding fragments of this type may be well expressed in bacterial, yeast, and mammalian cell systems. Such fragments may also be resistant to otherwise damaging conditions, such as freeze-drying or heat denaturation.

Antibodies may have both Variable and Constant domains. It will be appreciated that antigen-binding fragments (e.g. scFV antibodies) that comprise essentially the Variable region of an antibody without any Constant region are also encompassed by the present invention.

Purely by way of example, Fab, F(ab′)₂ and other immunoreactive fragments, can be obtained by digesting monoclonal antibody with a proteolytic enzyme which does not decompose the antigen-binding site (Fab), such as papain, pepsin or the like. The antigen-binding fragments thus generated may then be isolated and purified using routine techniques. Antigen-binding fragments having the specificity of monoclonal antibodies can be used in the same way as the monoclonal antibodies themselves in the method of this aspect of the invention. Antigen-binding variants also have the specificity of the monoclonal antibodies themselves, and may include further regions such as a reporter moiety, as discussed below.

As described above, SEQ ID NOs 1 to 6 provide the amino acid sequence of the variable regions of monoclonal antibodies derived from hybridoma cell lines KA1, 1B6 and 21C3. By “variant” of the monoclonal antibody, we also include those antibodies that have a variant of that polypeptide sequence but retain the antigen binding specificity discussed above.

The amino acid sequence provided in SEQ ID NOs 1 to 6 include the complementarity determining regions (CDRs) and the framework regions of the variable domains. Variants of that amino acid sequence may include alterations in the CDRs, or in the framework regions, as can be appreciated by the skilled person. Means of preparing variants of that polypeptide sequence are well known in the art; for example, recombinant nucleic acid technologies are provided in standard laboratory manuals such as Sambrook et al., Molecular Cloning. A laboratory manual. 2001, Cold Spring Harbour publications.

Monoclonal antibodies (including antigen binding fragments or variants thereof), in accordance with the invention may be used in a number of assays in which it may be beneficial to be able to obtain information as to the location or binding of the binding molecules. In such cases it may be preferred that the monoclonal antibodies be labelled using a reporter moiety. Such reporter moieties may be directly or indirectly linked to a monoclonal antibody or to the target antigen

Suitable reporter moieties will be well known to those skilled in the art, as will methods by which they may be attached to the monoclonal antibody. A suitable reporter moiety (label) may be selected from the group consisting of a fluorescent moiety; a luminescent moiety; a bioluminescent moiety; a radioactive material; a prosthetic group (such as biotin); a colorimetric moiety; a nanoparticles having suitable detectable properties, and a chromogenic moiety.

Purely by way of example, and without limitation, suitable fluorescent moieties may be selected from the group consisting of: fluorescein isothiocyanate (FITC); rhodamine (TRITC); phycoerythrin; allophycocyanin; coumarin (AMCA); Texas red; and cyanine (Cy2, Cy3 or Cy5). Other suitable fluorescent moieties that may be used in labelling monoclonal antibodies will be readily apparent to the skilled person.

Recently new technologies such as quantum dots have been discovered which afford highly efficient fluorescent properties.

A suitable labelling of monoclonal antibody with FITC may be achieved using the following protocol. The antibody is prepared as a 2 mg/ml solution in 0.1M sodium carbonate (pH 9.0). FITC is dissolved in DMSO at 1 mg/ml and added slowly to the antibody solution (to a concentration of about 50 μg of FITC per ml of antibody). The mixture of antibody and FITC is incubated in dark for 1-2 hours at room temperature. The labelled antibody may then be separated by gel filtration, affinity chromatography or buffer exchange.

For the purposes of the present disclosure, luminescent and bioluminescent moieties may be taken to encompass both moieties that have luminescent properties themselves, and moieties capable of giving rise to luminescent products. Luminol represents a suitable example of a luminescent moiety that may be used to label antibodies of the invention. Luciferase provides an example of a bioluminescent moiety (in this case a moiety able to generate a luminescent product) that may be used label antibodies.

Suitable radioactive materials that may be used to label antibodies will be apparent to the skilled person. Merely by way of illustrative example, suitable radioactive materials may include radioisotopes selected from the group consisting of: ¹²⁵I; ¹³¹I; ³⁵S; ³H; ¹⁴C; ³²P; ^{99m}Tc and ¹¹¹In.

An antibody may be labelled using prosthetic groups that bind one another with high specificity. An example of such a suitable prosthetic group that will be well known to those skilled in the art is biotin. An antibody may be labelled with biotin, and the biotinylated antibody exposed to a specific binding partner for biotin (for example avidin or streptavidin). The binding partner may carry a separate label, such as a fluorescent label.

Colorimetric moieties that may be used to label antibodies include, but are not limited to: colloidal gold; and coloured glass or plastic (e.g. polystyrene, polypropylene, latex, or the like) beads or quantum dots.

Examples of chromogenic moieties that may be used to label antibodies include chromogenic enzymes such as: horseradish peroxidise and alkaline phosphatase. Various substrates that may be used to generate detectable products representative of activity of these enzymes are well known, and indeed are commercially available from many suppliers of laboratory reagents.

In addition to the direct labelling, in which reporter moieties may be linked directly to the monoclonal antibody, indirect labelling techniques can be used. Methods by which antibodies may be indirectly labelled are well known to those of ordinary skill in the art.

One suitable manner by which antibodies may be indirectly labelled is by means of a “primary antibody”/“secondary” antibody strategy. Briefly, in such a strategy the unlabelled antibody is used as a “primary antibody” able to bind to FLC in a sample (e.g. a patient sample). A labelled “secondary antibody” (chosen to react solely with the antibody and not with other materials in the sample) is then used to bind to the primary antibody. Thus the unlabelled antibody is effectively bound to the label attached to the secondary antibody.

The labelling of antibodies (whether directly or indirectly) allows detection of these molecules. Typically unbound molecules carrying the chosen label (such as unbound directly labelled antibodies or secondary antibodies) will be removed from a sample so that substantially the only label remaining is associated with bound antibodies.

Detection of the label is then taken to represent detection of the bound binding molecules. The means by which the label will be detected will depend on the nature of the label selected. For instance, a fluorescent label will be detected by illuminating the sample (containing the bound antibody) with light at the excitation wavelength of the fluorescent label, and detecting for the presence of light at the emission wavelength of the selected fluorophore.

An antibody labelled with a chromogenic enzyme may be detected by incubating the sample (containing the bound antibody) with a chromogenic substrate of the selected enzyme, and detecting for the presence of the coloured product produced as a result of action of the enzyme on the substrate.

Ways in which antibodies that have been labelled using alternative reporter moieties may be detected will be well known to those skilled in the art.

The method of the first aspect of the invention can be considered to be an immunological assay in which the amount of free κ immunoglobulin light chain molecules and free λ immunoglobulin light chain molecules in a test sample are measured, and then from this the ratio of free κ to free λ levels can be determined. By way of example, such assays include ELISAs, nephelometry, turbidimetry and flow cytometry, and may use labelled beads such as Luminex™ beads or aluminum based particles, such as UltraPlex™ system from Pronostics™. Alternatively, a protein microarray based assay may be produced using the antibodies or light chains with the monoclonal antibodies in the fluid phase. Additionally using binding cassettes with a surface coated with the antigen or monoclonal antibody may be used. In this case binding determinants (surface plasmon reasonance) or optical changes such as linear or circular diachroism may be readouts of binding may be used to read the binding of the monoclonal antibodies to the light chains.

Methods of measuring the amount of a particular target molecule in a sample using a binding molecule that specifically binds to the target are well known in the art.

Preferably the ratio is determined by immunoassay, most preferably via an immunosorbent assay such as ELISA (Enzyme Linked ImmunoSorbent Assay). ELISA-type assays per se are well known in the art. They use binding molecules, including monoclonal antibodies or antigen binding fragments, to detect particular target molecules in samples. Such an assay can be used for the method of the invention.

ELISA uses binding molecules to detect target molecules. One or more of the antibodies used in the assay may be labelled to allow for detection of binding of the target molecule to the binding molecule; examples of such labels are set out earlier in the specification. The construction of ELISA-type assays is itself well known in the art. For example, a binding molecule specific for a FLC is immobilised on a substrate by methods which are well known in the art, and the substrate exposed to the test sample. FLC in the test sample are bound by the binding molecule. Unbound molecules may be washed away. In ELISA assays the presence of bound FLC may be determined by using a secondary labelled antibody specific to a different part of the FLC of interest than the binding molecule. Such “primary” and “secondary” systems are discussed further above. It is then possible from the amount of the “secondary” antibody to determine how much FLC was present in the sample. Thus using such a system it is possible to measure the amount of free κ immunoglobulin light chain molecules and free λ immunoglobulin light chain molecules in a test sample and therefore the ratio of free κ to free λ levels.

Flow cytometry can also be used to detect the binding of FLC to monoclonal antibodies and measure the ratio of free κ to free λ levels. Here an antibody is attached to a support material, such as a polystyrene or latex bead. The support material is mixed with the test sample and a second detecting antibody, such as antibody specific for free λ immunoglobulin light chain molecules. The detecting antibody is preferably labelled with a detectable label, which binds the FLC to be detected. This results in a labelled support material when the FLC to be assayed is present.

Labelled support material may then be detected via flow cytometry. Different labels, such as different fluorescent labels may be used for the monoclonal antibody that specifically binds to free κ to that used for the monoclonal antibody that specifically binds to free λ. This allows the amount of each type of immunoglobulin bound to be determined simultaneously and allows the rapid identification of the κ:λ ratio in a test sample. Different sized support material can also be used, or further support materials that are distinguishable from each other.

The method of the first aspect of the invention may also be a competition assay, that is to say a method of measuring the level of free κ immunoglobulin light chain molecules and free λ immunoglobulin light chain molecules in a test by employing a monoclonal antibody as provided herein in a competition assay.

Hence a preferred embodiment of the first aspect of the invention is wherein steps (i) and (ii) are performed in the presence of labelled κ and λ immunoglobulin light chain molecules that are capable of being bound by the monoclonal antibodies and are distinguishable from free κ and λ immunoglobulin light chain molecules in the test sample.

In this embodiment of the invention, steps (i) and (ii) of the method comprises measuring the amount of the labelled κ and λ immunoglobulin light chain molecules bound to the monoclonal antibodies. When a test sample is added to the assay method, any reduction in the amount of labelled κ and λ bound by the monoclonal antibodies is proportional to the amount of free κ and λ immunoglobulin light chain molecules in the test sample. In this way, it is possible to measure the amount of free κ and λ molecules and thus calculate the ratio of free κ and λ levels in the sample.

Methods for preparing labelled κ and λ immunoglobulin light chain molecules are provided in the accompanying example.

There are a number of different ways in which the labelled κ and λ immunoglobulin light chain molecules can be made distinguishable from the free κ and λ immunoglobulin light chain molecules in the test sample. However, a preferred embodiment of the invention is wherein the labelled κ and λ immunoglobulin light chain molecules are conjugated with a binding entity, preferably biotin. As can be seen in the accompanying example, this allows for the labelled κ and λ immunoglobulin light chain molecules to be detected using a Streptavidin-reporter conjugate.

In order to readily calculate whether there is any reduction in the amount of labelled κ and λ bound by the monoclonal antibodies, the method of the first aspect of the invention may include preparing a standard curve for the binding of the labelled κ and λ immunoglobulin light chain molecules bound to the assay reagent.

A preferred embodiment of the first aspect of the invention is wherein the monoclonal antibody that specifically binds to free κ is attached to a first support material, the monoclonal antibody that specifically binds to free λ is attached to a second support material, the first and second support materials being distinguishable from each other.

Multiplexing allows for simultaneous quantification of multiple analytes in one sample. They are a variety of different multiplexing systems available, including Luminex™ microbeads or aluminum based particles, such as UltraPlex™ system from Pronostics™. In such systems the monoclonal antibodies that bind to the analytes are attached to support materials that are distinguishable from each other. Such systems are well known in the art. An additional multiplexing system which could be exploited in measuring these analytes include protein arrays or microarrays, where different capturing monoclonal antibodies may be spotted or fixed at certain positions of either a well, chip or slide.

The accompanying examples provides a protocol for measuring the ratio of free κ immunoglobulin light chain molecules to free λ immunoglobulin light chain molecules in a test sample in which monoclonal antibodies are attached to differently fluorescently labelled Luminex™ beads, thus allowing the λ/κ ratio to be determined by the fluorescent wavelength. Luminex beads are available from Luminex Corporation, Austin, Tex., United States of America.

Hence a preferred embodiment is where the support material is microspheres, most preferably Luminex™ beads.

An alternative multiplexing system that can be used in the method of the invention is aluminum based particles, such as UltraPlex™ system from Pronostics™. Hence a further embodiment is wherein the support material is microscopic aluminium particles.

Steps (i) and (ii) of the method of the first aspect of the invention can be performed serially. That is, the amount of free κ in a test sample is measured, then the amount of free λ in a test sample is measured; or vice-versa. However, it is preferred that steps (i) and (ii) are performed simultaneously in parallel. Methods for the simultaneous measurement of free κ and free λ in a test sample include the use of the multiplexing systems discussed above. The accompanying examples provide a protocol for simultaneously measuring the amount free κ immunoglobulin light chain molecules and free λ immunoglobulin light chain molecules in a test sample.

As mentioned above, the method of the invention can be part of a multiplexing procedure which allows for simultaneous quantification of multiple analytes in one sample. Hence in addition to measuring the ratio of free κ immunoglobulin light chain molecules and free λ immunoglobulin light chain molecules in a test sample, in one embodiment the method further comprises measuring the amount of one or more further analytes in the test sample. For example, the analyte(s) may be albumin, whole immunoglobulins, CRP and/or B2m.

Preferably, the test sample is obtained from mammalian tissue or biological fluid, such as blood or serum from blood, or urine of a mammal, preferably a human.

A second aspect of the invention provides an isolated monoclonal antibody, or an antigen binding fragment or variant of said monoclonal antibody, that specifically binds to free κ, said monoclonal antibody comprising the amino acid sequence provided in SEQ ID NO:1 and/or SEQ ID NO:2. Preferably the monoclonal antibody or antigen binding fragment or variant is produced by hybridoma cell line KA1, deposited under Budapest Treaty at the ECACC under reference number 08071702.

A third aspect of the invention provides an isolated monoclonal antibody, or an antigen binding fragment or variant of said monoclonal antibody, that specifically binds to free κ, said monoclonal antibody comprising the amino acid sequence provided in SEQ ID NO:3 and/or SEQ ID NO:4. Preferably the monoclonal antibody or antigen binding fragment or variant is produced by hybridoma cell line 1B6, deposited under Budapest Treaty at the ECACC under reference number 08071704

A fourth aspect of the invention provides an isolated monoclonal antibody, or an antigen binding fragment or variant of said monoclonal antibody, that specifically binds to free λ, said monoclonal antibody comprising the amino acid sequence provided in SEQ ID NO:5 and/or SEQ ID NO:6 Preferably the monoclonal antibody or antigen binding fragment or variant is produced by hybridoma cell line 21C3 deposited under Budapest Treaty at the ECACC under reference number 08071701.

A fifth aspect of the invention provides an isolated monoclonal antibody, or an antigen binding fragment or variant of said monoclonal antibody, that specifically binds to free κ, said monoclonal antibody produced by hybridoma cell line B3B4, deposited under Budapest Treaty at the ECACC under reference number 08071705.

A sixth aspect of the invention provides an isolated monoclonal antibody, or an antigen binding fragment or variant of said monoclonal antibody, that specifically binds to free λ, said monoclonal antibody produced by hybridoma cell line 21E9, deposited under Budapest Treaty at the ECACC under reference number 08071703.

The second, third fourth, fifth and sixth aspects of the invention provide isolated monoclonal antibodies that can specifically bind with free κ or free λ immunoglobulin light chain molecules. Such molecules are discussed in detail in the first aspect of the invention.

By “isolated” we include where the binding molecule is present substantially free from any further polypeptides. Preferably any further polypeptide comprises less than 50%, 40%, 30%, 20%, 10%, 5%, 2%, 1%, 0.5%, 0.1%, 0.01% or less of the total polypeptide by weight.

Methods of preparing a binding molecule according to this aspect of the invention are well known and readily performed using standard molecular biology techniques as is well known to those skilled in the art and described further in Sambrook et al., Molecular Cloning. A laboratory manual. 2001. Cold Spring Harbour publications.

Monoclonal antibodies of the invention, however prepared, may be purified using well-known antibody purification techniques. Suitable examples of antibody purification technology that may be used to purify antibodies of the invention comprises precipitation (salting-out) with ammonium sulfate or the like, ion exchange chromatography using a diethylamino-ester (DEAE) derivative, a carboxymethyl (CM) derivative, or the like, hydroxyapatite chromatography, gel filtration chromatography, and affinity chromatography using Protein A or Protein G, among others, including binding to antigen against which the antibody has been raised. It will be appreciated that combinations of the techniques suggested above may be utilised in purification of the monoclonal antibody.

An example of a method for preparing a monoclonal antibody from a hybridoma cell line is provided in the accompanying examples.

A seventh aspect of the invention provides a support material having attached a monoclonal antibody, or an antigen binding fragment or variant of said monoclonal antibody, according to any of the second, third, fourth, fifth and sixth aspects of the invention.

Embodiments of the seventh aspects of the invention includes where the material is microspheres, preferably Luminex™ microspheres, or microscopic aluminium particles, preferably Pronostics™ UltraPlex™. Information concerning both such materials are provided above in relation to the first aspect of the invention. The accompanying examples provide further details as to how to prepare embodiments of these aspects of the invention.

Am eighth aspect of the invention provides hybridoma cell line KA1 with deposit reference 08071702 accession number.

A ninth aspect of the invention provides hybridoma cell line 1B6 with deposit reference 08071704 accession number.

A tenth aspect of the invention provides hybridoma cell line B3B4 with deposit reference 08071705 accession number.

An eleventh aspect of the invention provides hybridoma cell line 21C3 with deposit reference 08071701 accession number.

A twelfth aspect of the invention provides hybridoma cell line 21E9 with deposit reference 08071703 accession number.

Suitable techniques for the maintenance of hybridomas are well known to those skilled in the art. For example, hybridomas can be cultured and subcultured using known cell culture media, such as RPMI 1640 or Dulbecco's modified essential medium (DMEM). Samples of hybridomas produced in the manner described above, and producing the monoclonal antibody of the invention, may be preserved in liquid nitrogen prior to thawing for future use.

Hybridomas of the invention are suitable for culturing on a large scale, to facilitate the production of large quantities of the antibodies of the invention. Hybridomas of the invention may, for instance, be cultured in 15% fetal calf serum (FCS)-RPMI 1640, and monoclonal antibodies of the invention can be prepared from the culture supernatant.

As an alternative, hybridomas of the invention may be injected intraperitoneally into experimental mice, to form ascitic tumours. The monoclonal antibodies of the invention can then be prepared from the ascites fluid.

A thirteenth aspect of the invention provides a kit for performing a method of measuring the ratio of free κ immunoglobulin light chain molecules to free λ immunoglobulin light chain molecules in a test sample comprising:
(i) a monoclonal antibody, or an antigen binding fragment or variant of said monoclonal antibody, that specifically binds to free κ; and,
(iii) a monoclonal antibody, or an antigen binding fragment or variant of said monoclonal antibody, that specifically binds to free λ.

An embodiment of this aspect of the invention is wherein the monoclonal antibodies, or an antigen binding fragments or variants of said monoclonal antibodies, are attached to a support material.

A further embodiment of this aspect of the invention is wherein the kit further comprises labelled κ and λ immunoglobulin light chain molecules that are capable of being bound by the monoclonal antibodies and are distinguishable from free κ and λ immunoglobulin light chain molecules in the test sample. Means of preparing labelled κ and λ immunoglobulin light chain molecules are provided above and in the accompanying examples.

A further embodiment of this aspect of the invention is wherein the kit further comprises a data sheet aiding the calculation of the ratio of free κ to free λ levels in the test sample.

A fourteenth aspect of the invention provides a method of assessing the likelihood that a subject has, or may develop, a malignant plasma cell disease, an immunodeficiency, or to measure response of that disease to therapy, comprising determining the ratio of free κ immunoglobulin light chain molecules to free λ immunoglobulin light chain molecules in a test sample from the subject according to the method of the first aspect of the invention.

By “a malignant plasma cell disease” we include malignancies of plasma cells or B lymphocytes (multiple myeloma (MM), macroglobulinaemia, plasmacytoma, B-cell lymphoma, including B-cell non-Hodgkin lymphoma, B-cell chronic lymphocytic leukaemia), disorders involving monoclonal proteins of abnormal structure (primary systemic amyloidosis (AL), light chain deposition disease (LCDD), cryoglobulinaemia), and apparently benign, premalignant conditions (monoclonal gammopathy of undetermined significance (MGUS), smouldering MM). We also include plasma cell leukaemia and Waldenstrom's macroglobulinaemia.

Ratios of free κ to free λ levels in a test sample that are indicative of disease are discussed in, for example, Katzmann et al Clin Chem 2002;48:1437-1444. In this report the 95% reference interval for sfκ was 3.3-19.5mg/L and for sfλ was 5.7-26.3mg/L. The 95% reference interval for the serum free κ:λ ratio was 0.26-1.65.

The invention will now be further described with reference to the following Experimental Results and Figures in which:

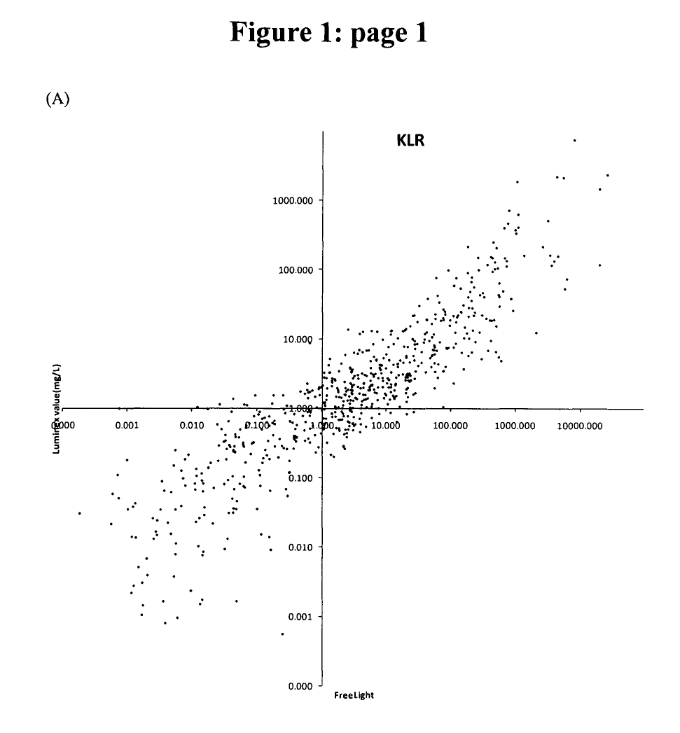

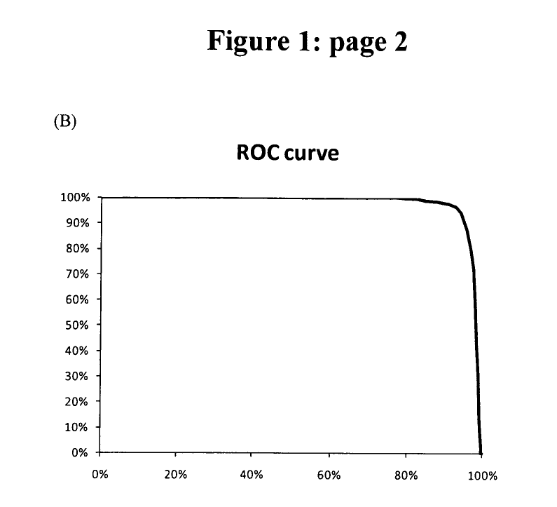

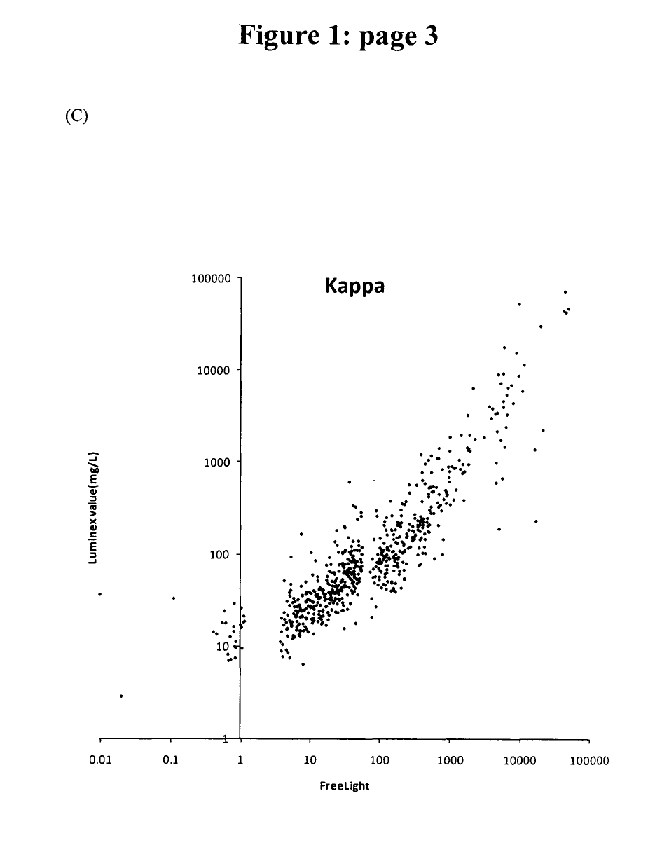

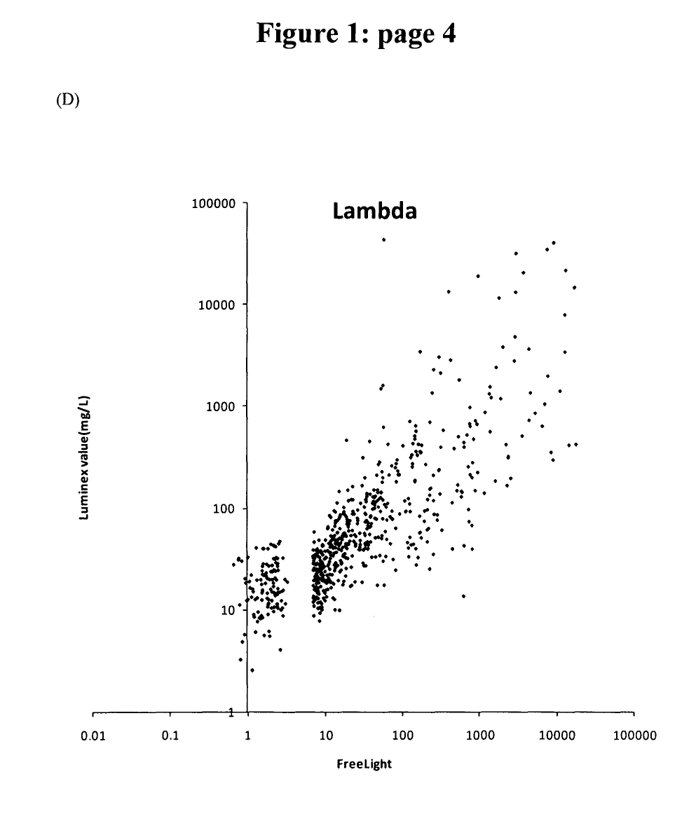

FIG. 1: A—Correlation of the κ:λ ratio between the Luminex assay (y) and Freelite (x). B—Receiver-Operator Characteristic of the Luminex assay in diagnosing monoclonal gammopathies (y-sensitivity; x-specificity). C, D—Correlation between sfκ sfλ respectively measured by Luminex (y) and Freelite (x) assays. Gaps in Freelite signal reflect multiple dilution intervals typical of the assay.

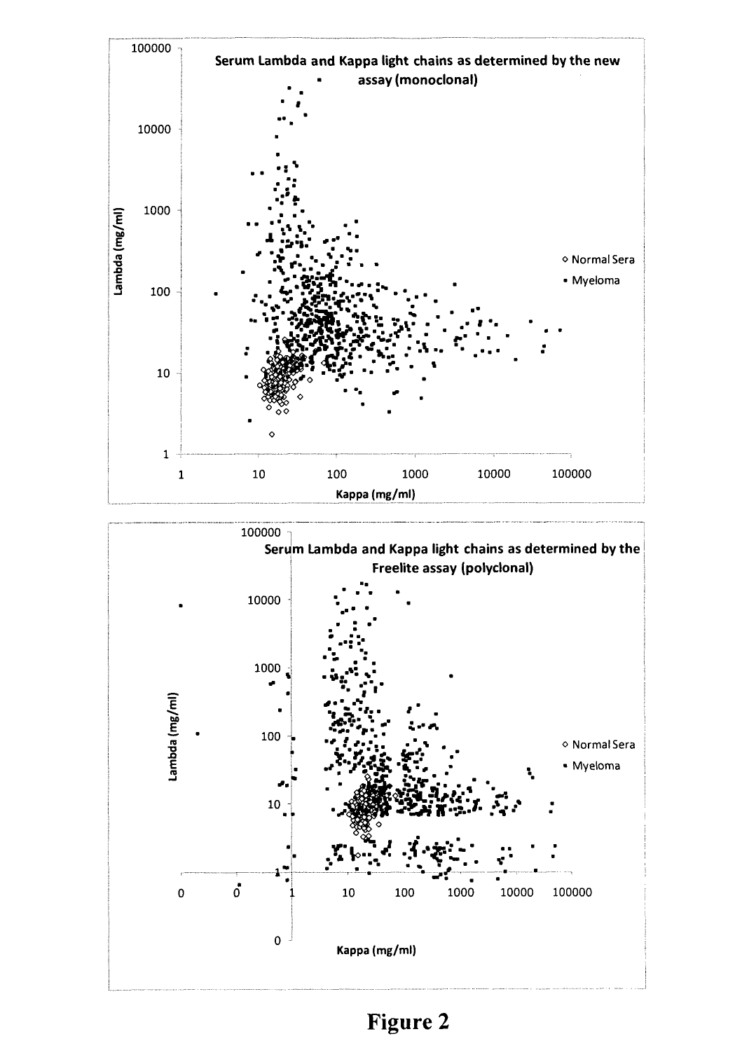

FIG. 2: ROC curve of Luminex Assay comparing 200 normal with 600 patient sera (FREELITE KLR vs Luminex KLR). Shows the distribution of 200 normal sera samples with 600 patient samples demonstrating distinct populations. Top=Luminex Assay; Bottom=FREELITE.

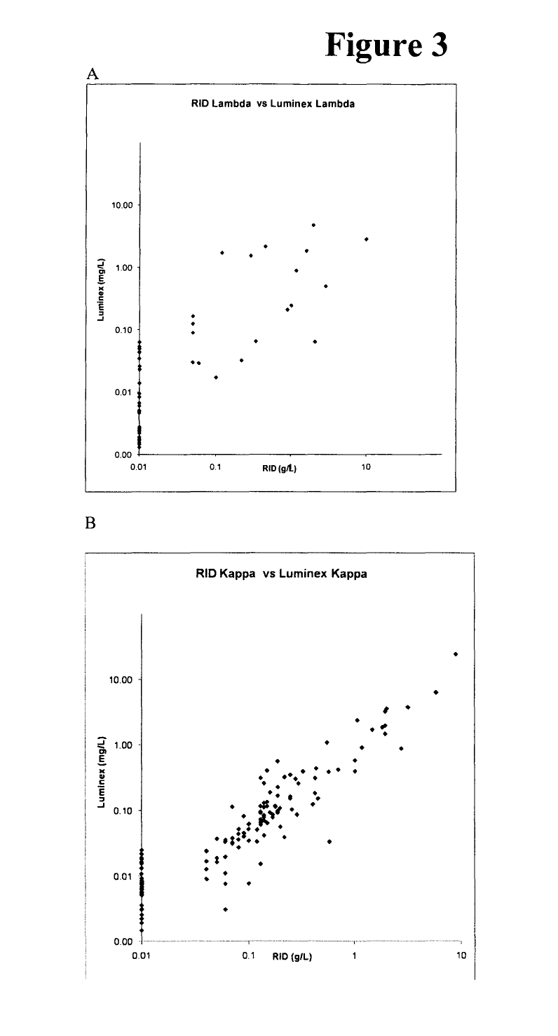

FIG. 3. Comparison of the measurement of urine free κ and λ by a flow cytometric (Luminex) based assay. (A) the correlation of urine free λ as measured by radial immunodiffusion (RID) using the 21C3 and 21E9 monoclonal antibodies. (B) the correlation of urine free κ as measured by RID using the KA1 and B3B4monoclonal antibodies.

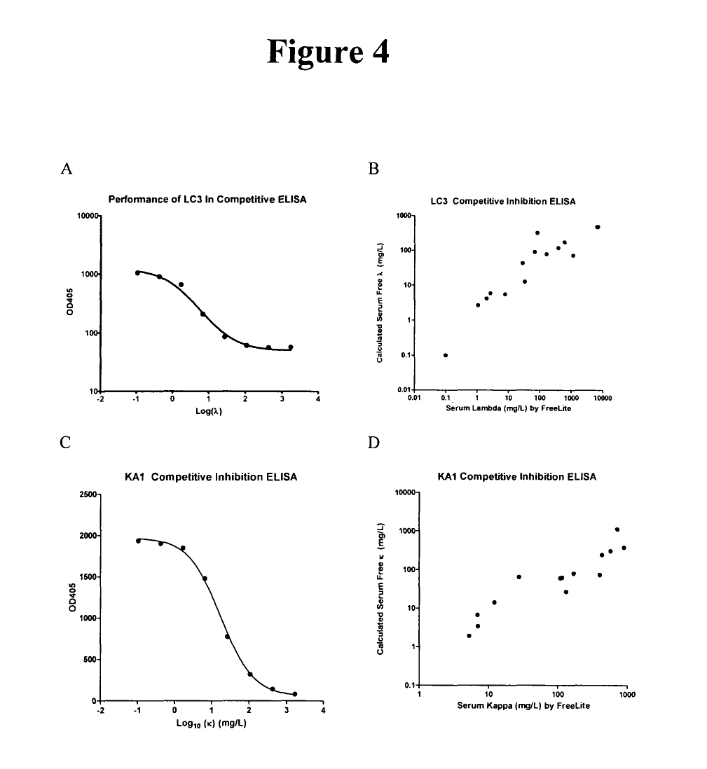

FIG. 4. The utility of using KA1 mAb to measure free κ and 21C3 to measure λ in the serum by a competitive inhibition assay. (A) Standard curve generated by adding in free λ from 1750mg/L to 0.1 mg/L displayed as log₁₀ values. The assay observes one-binding site competitive inhibition kinetics and thus the standard curve fits the equation: Concentration=IC₅₀×(MaxOD₄₀₅−ValueOD₄₀₅)/ (ValueOD₄₀₅−MinOD₄₀₅) where IC₅₀ is a constant representing the concentration of free κ or λ that gives a response half way between the maximum and minimum inhibition. MaxOD₄₀₅ and MinOD₄₀₅ are the maximum and minimum absorbances respectively at a wavelength of 405 nm. (B) Shows the correlation between Freelite™ and results derived from the competitive ELISA for the 21C3 antibody. (C) and (D) are the same data for KA1 antibody measuring free K in serum samples.

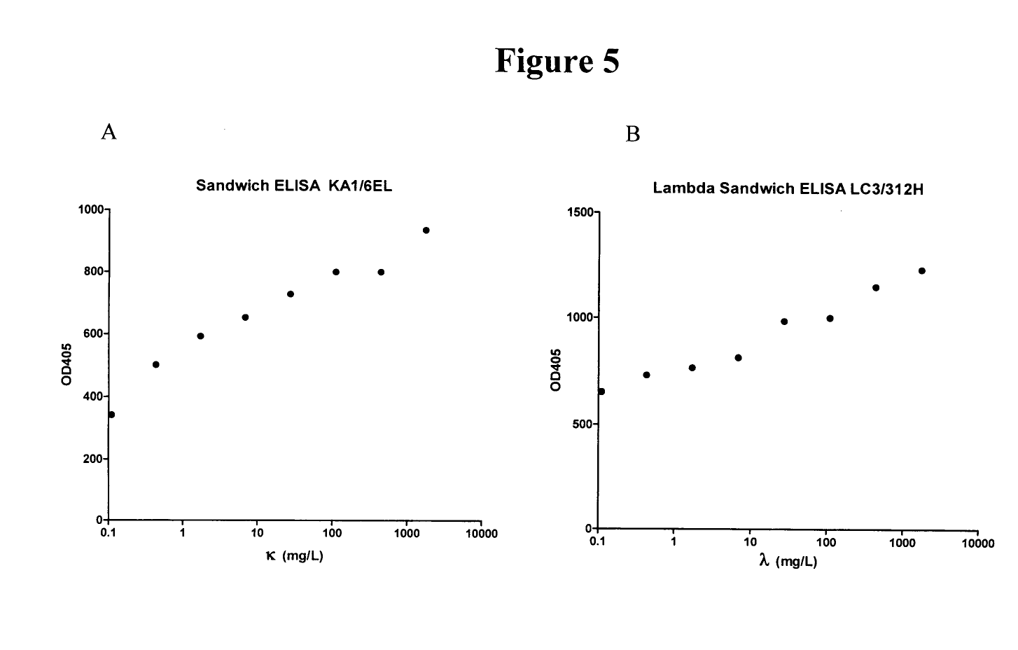

FIG. 5. The utility of using KA1 mAb to measure free K and 21C3 to measure λ in the serum by a sandwich ELISA.

(A) Standard curve generated by adding in free κ from 1750 mg/L to 0.1 mg/L displayed as log₁₀ values. KA1 is coated onto plates at 1 μg/ml and 6EL is used as the detection antibody. The assay observes one-binding site kinetics Y=Bmax*X/(Kd+X)+NS*X+Background (where Bmax=is the maximum specific binding; Kd is the equilibrium binding constant; NS is the slope of nonspecific binding; and Background is the amount of nonspecific binding with no added ligand). (B) Demonstrates the same results measuring λ using 21C3 coated onto plates at 1 μg/ml and 312H used as the detection antibody.

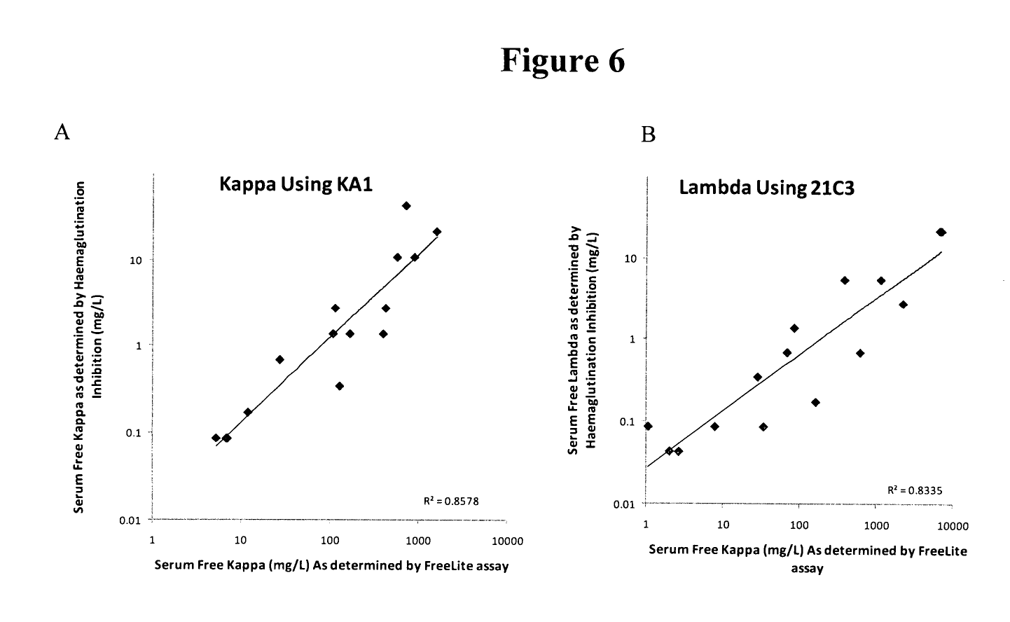

FIG. 6 The utility of mAbs KA1 and 21C3 to measure serum free light chains using an haemagglutination inhibition assay. Sheep red blood cells were coated with free kappa light chains and free lambda light chains respectively. The purified mAbs were titrated in doubling dilutions in RPMI+2% FCS and the end point determined at which agglutination was observed. This minimal concentration at which haemagglutination was observed was termed the minimal haemagglutinating dose (MHD). Stock solution of mAb at a concentration of twice MHD (2×MHD) was made up and checked for Haemagglutination. The test sera were first diluted 1:10 and subsequently doubly diluted in 25 μl volumes of RPMI+2% FCS. To each well 25 μl of diluent containing 2×MHD of mAb and then to each well 25 ul of coated sheep red blood cell suspension. Plates were left for 1 hour at room temperature for the red cells to settle. Agglutination was determined via macroscopic visualisation and the titre was determined where agglutination first observed (ie where agglutination was not inhibited). Patient sera were analysed in parallel with known standards and the concentration was determined and plotted against results as obtained via the FREELITE assay. The correlation between (A) using KA1 to measure free-K in serum samples via HIA (B) using 21C3 to measure free-λ in serum samples.

### EXAMPLE 1

### Materials and Methods Used in the Methods of the Invention

The method of the invention can use multiplexed flow cytometry to quantitate the level of free light chain in a clinical sample. Microspheres are conjugated with monoclonal antibodies specific for either free κ or λ. The assay described in the accompanying examples is a competitive inhibition assay where a fixed concentration of labelled (biotinylated) free κ and λ is mixed to a clinical sample and added to a mixture of beads each coated/conjugated to single monoclonal antibodies (anti-free κ and anti-free λ).

Set out below are instructions for the preparation of reagents (including MAb preparation) and methods that can be used during the method of the invention.

A. Buffers and Preservatives

1 Phosphate Buffered Saline (PBS). Dissolve 1 phosphate buffered saline Dulbecco A tablet (Oxoid, UK BR0014G) in 100 ml distilled water. Check is pH 7.4+/−0.2 on a pre-calibrated pH meter.

2. 10% Azide Solution.

With care, weigh out 10 g sodium azide (Sigma S2002) and dissolve in 100 ml distilled water 0.2 μm filter.

3. 1 M sodium hydroxide (1M NaOH): 400 g sodium hydroxide pellets (Sigma S-5881) in 10 ml distilled water. Mix well and carefully.

4. Activation Buffer.

Dissolve 3 g Sodium phosphate monobasic (NaH₂PO₄; Sigma S0751) in 250 ml distilled water adjust pH to 6.2 with 1M NaOH.

5. Wash and storage buffer. (PBS 1% BSA 0.05% Tween20 0.1% sodium azide) 200 ml PBS (see 1. above) containing:

a) 2 g bovine serum albumin (BSA; Sigma A4503)—source of BSA is important

b) 100 ul Tween 20 (polyoxyethylene sorbitan monolaurate; Sigma P.1379)

c) 2 ml 10% Azide Solution (see 2. above) 2 ml 10% stock solution.

Mix well and adjust pH to 7.4 with 1M NaOH (see 3. above) 0.2pm filter store at 2-8° C.

6. Luminex Buffer (LB Buffer). (PBS 0.2% BSA 0.05% Tween 20 0.05% NaN₃)

PBS (see 1.above) 1 Litre containing:

2 g BSA (see 5a.above) 2 g/L

500 μk Tween20 (see 5b.above)

5 ml 10% Azide Solution (see 2. above).

Adjust pH to 7.4 with 1M NaOH (see 3. above) 0.2 μm filter store at 2-8° C.

7. Dilution Buffer.

200 ml PBS (see 1.above) containing

1% BSA (see 4a.above) 2 g

0.1% sodium azide (see 2.above) 2 ml 10% stock solution.

Adjust pH to 7.4 with 1M NaOH (see 3. above) 0.2 μm filter store at 2-8° C.

8. Benzamidine. (Protease Inhibitor)

Prepare 100×stock solution 10% w/v in distilled water:

Add 1 g Benzamidine (Sigma B2009) in 10 ml distilled water and 0.2 μm filter sterilised. Aliquot into 5 ml in sterile glass bottles and the headspace spurged with nitrogen store at 2-8° C. in tightly stoppered containers spurged with nitrogen

Add 1 in 100 and re-spurge the headspace of the stock bottle.

9. ε Amino Caproic Acid (EACA) Protease Inhibitor

Prepare 100×stock solution 10% w/v in distilled water:

Add 1 g EACA (Sigma A7824) in 10 ml distilled water and 0.2 μm filter sterilised.

Aliquot into 5 ml in sterile glass bottles store at 2-8° C.

B. Coupling of Antibody to Luminex Microspheres.

Wherever possible, maintain sterility.

1. Prepare PBS, activation, wash and storage buffers.

2. Select the region number of the carboxylated microsphere to be used: the company supplies 100 ‘regions’. Ensure that there is no coincidence with other microsphere especially when using multiplex analysis. Preferably each antibody is coated onto its own microsphere region.

3. Vortex the container of microsphere for 30 secs at the maximum speed of 2500/min (MS 1 minishaker) followed by sonication for 1-3 mins in an Ultrawave sonicator to disperse the microsphere pellet.

4. Pipette 200 μl each microsphere type (1.25×10⁷ microspheres/ml) into separate wells of a 96 well filter plate (Millipore Multiscreen HTS): this will be enough for at least 50 tests. If want enough for 100 tests, use 2 wells of 200 μl microsphere s each.

5. Aspirate the fluid from the microsphere s on the vacuum manifold (Millipore, part number MSVMHTS00) fluid is pulled through the bottom of the wells leaving the microsphere s on the filter mesh.

6. Add 200 μl activation buffer to each well, suck through and repeat. Leave in a 3^{rd} wash of 200 μl until step 10.

7. Prepare a fresh solution sulpho-NHS.

Weigh out approx. 5 mg sulpho-NHS (N-hydroxysulfosuccinimide sodium salt; Sigma #56485) and dissolve in 100 μ; of activation buffer to give 50 mg/ml solution.

8. Prepare a fresh solution EDC.

Weigh out 5 mg EDC (1-ethyl-3-(3-dimethylaminopropyl)carbodiimide hydrochloride; Sigma E6383) and dissolve in 100 μl of activation buffer to give 50 mg/ml.

9. Prepare coupling buffer

In 2 ml sterile microtubes with screw cap (Sarstedt #72.694) mix 10 μl EDC solution, 10 μl sulpho-NHS solution and 80 μl activation buffer (scale up for coupling more than one microsphere)

10. Aspirate the activation buffer from the microspheres using the vacuum manifold. Add 100 μl coupling buffer (see 9 above) to each well of microspheres in the filter plate.

11. Shake for 30 mins at RT in the dark by covering the plate with the lid. (Shaker-Labsystems Wellmix speed 7-8).

12. Using the vacuum manifold aspirate off the coupling buffer on the vacuum manifold. Wash microsphere s with 200 μl PBS 3 times.

13. Dilute each antibody with PBS to 1 mg/ml.

14. Add 100 μl antibody to the appropriate well. Shake in the dark for 2-3 hours at room temperature.

15. Wash wells with 200 μl storage buffer 3 times (using vacuum manifold).

16. Vigorously resuspend the microsphere s in 200 μl storage buffer and transfer to sterile 2 ml microtube with screw cap. Repeat twice more (600 μl in total) so that there are no microsphere s left in the well. Approximate beads concentration will be 4×10⁶/ml.

17. Store at 2-6° C. Must not be frozen. Can be stored up to 6 months.

C. Preparation of FLC Proteins

Free light chain (FLC) proteins are prepared from urine or dialysates of patients with myeloma resulting in Bence Jones (BJ) proteins in the urine. Urine or more often the dialysate from renal dialysis is collected aseptically in 5 L plastic bags with Luer lock fittings especially designed for the dialysis equipment. To minimise the growth of micro-organisms, sodium azide can be introduced into the bag to give a final concentration of 0.1%. For 5 L, this would be 50 ml of 10% stock solution (see buffers section).

The bags are stored in the cold room (2-6° C.) in plastic boxes with a lid. 5 ml samples are taken from each bag identifiable by the number of the bag and letter and number code for the source of the dialysate. The samples are assayed for the type of BJ FLC and the concentration using the assays routinely used in the clinical laboratory. Those bags with a high concentration of FLC with a minimum concentration of the alternative FLC, are retained and those not required, are disposed of.

The samples are assayed by SDS PAGE to determine the extent and nature of any other proteins present such as albumin.

The BJ protein is isolated using High Performance Liquid Chromatography (HPLC) programmed on an AKTA purifier machine. The resultant protein is dialysed against distilled water and freeze dried. The powder is stored in screw capped bottles at 2-6° C.

Preparations of protein for biotinylation or for the calibration curve are prepared from at least 4 and preferably 5 freeze dried preparations of each FLC type. Each protein is prepared separately and then pooled to give both kappa type and lambda type at a final concentration of 7 mg/ml.

1. 100 mg of each freeze dried protein is weighed out and added to 10 ml filter sterile saline in sterile 30 ml conical universal tubes. Without mixing, the freeze dried material is left in the fridge to go into solution overnight.

2. The mixture is mixed gently and then centrifuged for 5 mins at 3000 rpm on a bench centrifuge.

3. The supernatant is passed through a 0.2 μm filter into a sterile tube.

4. A 50 μl sample is assessed for concentration of protein after diluting 1 in 10 in saline. Measurement is by spectrophotometer set at a wavelength of 280 nm. The calculation of concentration uses an extinction coefficient of 1.18 (Practical Immunology, 4th edition. By Frank C. Hay, Olwyn M. R. Westwood. Wiley-Blackwell 2002).

5. The proteins need to be mixed to give equal amounts of each preparation. There may be some loss of material due to insolubility after the freeze drying process. Thus having determined the concentration of each protein preparation, the volume of each preparation is calculated to give, for example, 80 mg of each protein. For each FLC type, the individual preparations are mixed, the concentration determined and adjusted with the addition of PBS to give 7 mg/ml. Finally the preparation is refiltered through 0.2 μm sterile filter and allocated a batch number. Preparations for biotinylation must not have any preservative added and be biotinylated within 48 hrs. Preparations for calibration curves can be stored with the addition of azide (0.09%) and frozen at −20° C. or −80° C. Once thawed, shelf life can be extended with the addition of benzamidine and EACA each at a final concentration of 0.1%.

D. Biotinylantin of Protein. (Including Kappa and Lambda Light Chains)

The biotinylated proteins have a useable “shelf life” of 6 months, upon addition of 0.1% azide.

Reagents

1 M Bicarbonate Buffer pH 8.3

Sodium bicarbonate (sodium hydrogen carbonate NaHCO₃; Serva Electrophoresis research grade #30180). Weigh out 21 g sodium bicarbonate and dissolve in 200 ml distilled water with Teflon-coated flea on magnetic stirrer. Adjust pH to 8.3 with 1M NaOH and make up to 250 mL.

Free Light Chain (FLC) Preparations (See Standard Operating Method) in PBS.

Kappa FLC at 7 mg/ml.

Lambda FLC at 7 mg/ml.

Preparations must not contain free amine containing compounds (such as Tris) as they will compete for conjugation with the amino-reactive compound. Low concentrations of sodium azide (<3 mM) or thiomersal (<1 mM) will not interfere with the reaction but it is preferable to be without.

Biotin amidohexanoyl-6-aminohexanoic acid N-hydroxysuccinimide ester (biotin NHS) minimum 95% TLC (Sigma B3295)

Dimethyl sulfoxide (DMSO) minimum 95% GC (Sigma D5879).

Biotinylation Method

1). Add 40 μl 1M bicarbonate buffer to 400 μl of each FLC preparation in a 2 ml microtube (e.g. Eppendorf) to bring the pH to 8.3.

2). Dissolve biotin NHS in DMSO by adding 200 μl DMSO to 2 mg biotin NHS by mixing well.

3). Add 50 μL of the biotin NHS in DMSO to each FLC preparation and mix for 1 hour at RT on a rotary mixer.

4) Prepare buffer exchange. (New concentration FLC=5.71 mg/ml)

Buffer Exchange.

Columns: NAP™ Sephadex G-25 DNA Grade: (GE Healthcare 17-0853-01). Place each column in the rack on the collection tray and remove caps top and bottom. Equilibrate each column by passing 10 ml of PBS through allowing it to drip into the tray. When PBS is level with the top of the gel, add 490 μl of biotinylated FLC preparation, allow this to pass through the gel. Position column over the top of a sterile microtube for collection of run-through. Add 1 ml PBS to gel and collect run-through until PBS is level with the top of the gel. Measure volume of run-through and calculate the new concentration of the buffer exchanged biotinylated κ-FLC or λ-FLC (e.g. for 490 μl 5.71 mg/ml collected as 1 ml fraction final concentration now 2.8 mg/ml).

Measurement of Efficiency of Biotinylation.

Mix together in a 1 ml microtube,

30 μl of each type of coated microsphere (anti-κ and anti-λ microspheres have different identification region number) i.e. 60 μL altogether make up to 800 μl with storage buffer.

E. Measurement of Efficiency of Coupling of Monoclonal Antibody to Microspheres.

1. Mix together in 2 ml microtube, 3 μl of each conjugated microsphere (60 μL altogether) make up to 800 μl with storage buffer (i.e. 740 μl)

2. Add 50 μl of Luminex buffer (LB) to one well then aspirate on the vacuum manifold—to wet the well.

3. Add 50 μl of goat anti-mouse PE reagent (Invitrogen P-852) diluted 1:500 in dilution buffer to each well.

4. Add 10 μl of microsphere mixture to the well.

5. Shake in the dark for 30 mins.

6. Wash well 3 times with 200 μl LB buffer each time aspirating by vacuum.

7. Add 130 μl LB buffer to well and shake 30 secs.

8. Load plate into the Luminex 100 bioanalyser and run.

F. Kappa and Lamba FLC Competitive Assay

Standard curve consists of 8 points: 1750 mg/L; 440 mg/L; 110 mg/L; 27 mg/L; 6.8 mg/L; 1.7 mg/L; 0.4 mg/L; 0.1 mg/L.

1. In a 1 ml tube, microsphere mix:
30 μl of each type of coated microsphere identified by a different region number for anti-κ conjugated and anti-λ conjugated microspheres.

Make up to 800 μl with storage buffer—final concentration 1.5×10⁵ microspheres/ml for each microsphere thus total microsphere concentration will be higher if more than one microsphere will be used in the assay.

2. Dilute κ-FLC and λ-FLC standard curve protein from 7 mg/ml stock in 4 fold dilution curve such that first standard has κ-SFL at 1750 mg/L and λ-SFL at 1750 mg/L (i.e. Mix 30 μl κ-SFL at 7 mg/ml+30 μl λ-SFL at 7 mg/ml+60 μl dilution buffer to make 120 μl of upper standard−1750 mg/L. Take 30 μl of this and add to 90 μl of dilution buffer to generate serial dilutions from 440 mg/L; 110 mg/L; 27 mg/L; 6.8 mg/L; 1.7 mg/L; 0.4 mg/L down to 0.1 mg/L) to give 90 μl of each dilution. It is recommended that the standard curve is run in duplicate and thus a total of 2×40 μl=80 μl of each standard will be needed.

3. Dilute biotinylated proteins (at 2.8 mg/ml) 1/1000 in dilution buffer in 1 ml tubes.

4. Prepare a 96 well filterplate by wetting wells with 50 μl of Luminex buffer and aspirating using a vacuum manifold.

5. Add reagents to each well as follows:
Standard Curve:
   400 Standard κ/λ dilution (from step 2)
   10 μl Biotinylated Kappa
   10 μl Biotinylated Lambda
   60 μl Luminex buffer
Samples:
   40 μl Each sample
   10 μl Biotinylated Kappa
   10 μl Biotinylated Lambda
   60 μl Luminex buffer

6. Shake in dark for 5 minutes

7. Add 10 μl of mixed microspheres (from step 1) to each well.

8. Incubate in the dark for 30 minutes.

9. Wash 3 times with 200 μl Luminex buffer, aspirating using the vacuum manifold.

10. Add 50 μl of 2 μg/ml Streptavidin-Phycoerythrin (SA-PE) to each well.

11. Incubate in the dark for 30 minutes.

12. Wash 3 times with 200 μl Luminex buffer each time and aspirating.

13. Add 130 μl Luminex buffer to each well

14. Shake in the dark for 30 seconds.

15. Load plate into the Luminex 100 bioanalyser and run.

G—Sample Preparation

The following protocols set out general procedures for preparing serum and urine samples suitable for use in the method of the invention.

Serum

Most specimens of whole blood taken by phlebotomists or nurses, arrive in 6 ml “Vacuette” containers which may contain Z serum clot activator (this accelerates clotting but has no effect on the resultant serum).Some specimens come in 20 ml universal containers or 9 ml “Sarstedt” monovettes.

Each specimen is assigned a laboratory number: this number is printed as a barcode and applied to the request form and the matching printed label is stuck onto the blood specimen container. There may be enough serum for several 2 ml containers; all of which receive the identical number. The request forms are booked in (patient details, test and assigned lab number entered onto a telepath database) and then scanned to keep an electronic copy indefinitely. The booking in procedure generates a printed barcode label which is applied to the 2 ml azide tubes into which the serum can be pipetted. This barcode tells the analysers and/or sample loaders, which assays to perform.

The blood in the original container is centrifuged for 5 mins at 3,500 rpm in an Heraeus Instuments megafuge 1.0 R at room temperature (approx. 22° C.) which separates red cells from the serum.

The serum is removed by sterile plastic Pasteur pipette (pastette) into a 2 ml screw cap tube containing dry sodium azide powder. (25 uL of 5M azide [32.5 g sodium azide in 100 mL distilled water] is pipetted into 2 ml screw cap tubes (caps left very loose) and left in the warm room (37 C) for a week for the water to evaporate fully. The percentage of azide, therefore, varies according to the serum volume added as not all specimens produce 2 ml of serum).

The specimens are ready for testing; they are stored at 4° C. until needed by the appropriate testing bench. The tubes are removed, assayed and replaced, usually within a 24 hr period and usually within 72 hrs of the specimen being booked in.

They are stored for 1 calendar month at 4° C. in a refrigerator. After 1 month, the specimens are “archived” into appropriate racks and stored at −20° C. in freezers for a minimum of 2 years. They may be kept longer if they are of special clinical interest.

Urine

Urine arrives in 30 ml universal containers. The specimen is usually taken from the first urination of the day into the containers containing 25 ul of 5M azide (as above) in solution. A lab number is assigned and the specimen booked in as for serum.

2 ml of the urine is pipetted into azide tubes (containing 25 ul 5M azide and allowed to dry) and barcoded as generated by the booking-in process. The number of barcodes generated reflects how many tubes are needed.

The remaining urine in the universal containers is refrigerated for 1 month at 4° C. then discarded if of no further interest. The 2 ml tubes are also stored refrigerated for 1 month before storing indefinitely at −20° C.

H—Anti Kappa FLC Monoclonal Antibody KA1—Details and Preparation

KA1 is a murine monoclonal antibody specific for human free kappa light chains i.e. NOT bound to the heavy chain of immunoglobulins of any class. It was raised by fusion of mouse balb/c spleen cell with P3-NS1/Ag-1 immortal partner.

Immunogen: CL-Cγ1 fragment of human IgG

Mouse IgG class; IgG1 isotype.

Specificity determined by Heamagglutination (HA) and Haemagglutination Inhibition (HAI) of sheep red blood cells coated with purified human serum proteins. (see Ling, Bishop and Jefferis 1977 J. Immunol. Methods.15:279).

Human IgG, IgA, IgM, IgD and FLC preparations used to confirm the specificity. Also confirmed using an ELISA (Enzyme Linked ImmunoSorbent Assay).

Original Specificity Determination:

Haemagglutination Inhibition (HAI) (Ling, Bishop and Jefferis {1977} J. Imm. Methods 15: 279-289. Use of antibody coated red cells for the sensitive detection of antigen and in rosette tests for cells bearing surface immunoglobulins).

Sheep RBCs are covalently linked with BJ kappa protein Ke using Chromium chloride. The monoclonal antibody is titrated (25 uL volumes in a 96 round bottomed well plate) against these coated RBCs to determine the concentration of MAb which will still give obvious agglutination. This is the minimum haemolytic dose (MHD). A stock of antibody at a concentration of 2MHD in diluent (2% FBS in RPMI 1640 medium) is made up. The antigens ie human serum immunoglobulins, are diluted (doubling dilutions) in diluent 2% FBS in RPMI up to 11 wells with the twelfth well containing diluent only as a control. 25 ul of diluent containing the MAb (at a concentration of 2 MHD) is added to each well. 30 uL of the RBC suspension is added to each well and left to settle at room temperature or in the fridge overnight. If the MAb binds the test antigen, then it is no longer available to agglutinate the RBCs and the RBCs will settle into a button on the base of the well. As the antigen becomes increasingly dilute, more of the MAb remains free to agglutinate the RBCs which should be obvious in the control well without antigen.

Results: MAb dilution 1/100,000 vs BJ kappa Ke

| | | |
|---|---|---|
| IgG1 kappa Bo | 5 | free kappa in the preparation |
| IgG1 kappa Re | 0 | |
| IgG1 kappa Cr | 0 | |
| IgG2 kappa Mi | 0 | |
| IgG2 kappa Ca | 0 | |
| IgG2 lambda M3 | 0 | |
| IgG2 lambda Jo | 0 | |
| IgG3 kappa Pi | 0 | |
| IgG3 kappa Gi | 0 | |
| IgG3 lambda Re | 0 | |
| IgG4 kappa Jon | 0 | |
| BJ kappa Bo | 11 | |
| BJ kappa Ne | 12 | |
| BJ kappa Na | 10 | |
| BJ kappa Ke | 12 | |
| BJ kappa Ho | 10 | |
| BJ kappa Re | 11 | |
| BJ lambda Ev | 0 | |
| Polyclonal IgG | 0 | |
| Fab | 6 | |
| F(ab)2 | 10 | |

Production:

Cells were accessed from storage in liquid nitrogen and grown in RPMI 1640 containing penicillin (120 ug/ml) and streptomycin (100 units/ml) and supplemented with 10% fetal bovine serum. Productivity and specificity was confirmed by ELISA and HA. Cells were bulked up for loading a MiniPerm bioreactor (Greiner Bio-One) and loaded at 5×10⁷ cells. The medium in the reservoir was RPMI 1640 with penicillin and streptomycin and 1 g/L extra glucose. The production module medium was the same supplemented with 10% fetal bovine serum which had been depleted of most of the bovine IgG by passing down an SpA column. Once established, the bioreactor was harvested every 2 days by removing the cell suspension with a syringe. The cells were spun down (1500rpm for 3 mins), the supernatant decanted and the cells resuspended in fresh medium and returned to the production module. The supernatant was passed through a 0.2 um filter before storing at −20° C. or purifying.

Purification:

(see SOPs for buffer recipes for protein purifications and chromatography methodologies)

Protein G coupled to Sepharose 4 fast flow—GE Healthcare 17-0618-01 5 ml

Equilibration/wash buffer—PBS pH 7.2 (sodium salts)

Elution buffer—0.1M citrate pH 2.4-2.6

Neutralisation buffer—1M TRIS HCl pH 8.8

Column storage buffer—PBS/Azide (0.1%)

Monoclonal antibody presented in culture medium as cell supernatant:

Check the pH and, if necessary, adjust to pH 7.2 with 1M HCl or 1M NaOH as appropriate. 0.2 u filter. Estimate the concentration of MAb in the supernatant (based on assay titre and previous purifications). Pipette the required volume of supernatant into a sterile container.

Wash the column with PBS, at least 3 bed volumes, and establish base line.

Load column with supernatant and collect last third of the volume loaded in case of overload and to monitor the efficiency of retrieval of the MAb (ELISA or HA assay). Wash with PBS and collect washings as some leaching may take place especially if the column is nearing its capacity.

Elute with acid: collect in glass tubes starting with 50 ul neutralisation buffer in tubes. Begin collection when chart recorder indicates concentration of eluate is approaching 0.2 mg/ml. To 300 ul of eluate add 50 ul neutralising buffer. Continue in like manner collecting in series of tubes (approx.5 ml per tube) as required. Mix. Continue until concentration falls to below 0.5 mg/ml. and collect “tail” in a separate tube. Quickly check the pH of each tube and add neutralising buffer to achieve a pH of at least 6.5 but no more than 6.8.

When base line is reached, wash with PBS for another run or wash with PBS/azide for storage of the column at 2-8° C.

Check breakthrough and washings for MAb content (see SOPs for antibody assays). Pass down column again if worthwhile.

Determine the concentration of protein in each tube by spectrophotometry at 280 A and a coefficient of extinction of 14.3.

Pool appropriate tubes to give a concentration of at least 1 mg/ml

Dialyse eluate against PBS (50 to 100 volumes of dialysis buffer) for at least 4 hrs on a magnetic stirrer and/or overnight at 2-8 C. (see SOP for dialysis)

Filter sterilise (0.2 u) (see SOP) purified MAb and take 50 ul for concentration determination by spectrophotometer (see SOP for protein concentration determination) and purity assays (ELISA, SDS PAGE etc). Minimum concentration is 1 mg/ml.

Aliquot 0.5 ml for quality control. Use to “coat” beads for Luminex assay and determine the efficiency of coating. Determine the binding of biotinylated FLC to the coated beads and obtain a standard curve.

I—Anti Kappa FLC Monoclonal Antibody 1B6—Details and Preparation

Murine monoclonal antibody specific for human free kappa light chains i.e. NOT bound to the heavy chain of immunoglobulins of any class. Raised by fusion of mouse balb/c spleen cell with P3-NS1/Ag-1 immortal partner.

Immunogen: CL-Cγ1 fragment of human IgG

Murine IgG class; IgG1 isotype.

Specificity determined by Heamagglutination (HA) and Haemagglutination Inhibition (HAI) of sheep red blood cells coated with purified human serum proteins. (see Ling, Bishop and Jefferis 1977 J. Immunol. Methods.15:279). Human IgG, IgA, IgM, IgD and FLC preparations used to confirm the specificity.

Also confirmed using an ELISA (Enzyme Linked ImmunoSorbent Assay).

Original Specificity Determination:

Haemagglutination Inhibition (HAI) (Ling, Bishop and Jefferis {1977} J. Imm. Methods 15: 279-289. Use of antibody coated red cells for the sensitive detection of antigen and in rosette tests for cells bearing surface immunoglobulins).

Sheep RBCs are covalently linked with BJ kappa protein Bo using Chromium chloride. The monoclonal antibody is titrated (25 uL volumes in a 96 round bottomed well plate) against these coated RBCs to determine the concentration of MAb which will still give obvious agglutination. This is the minimum haemolytic dose (MHD). A stock of antibody at a concentration of 2MHD in diluent (2% FBS in RPMI 1640 medium) is made up. The antigens ie human serum immunoglobulins, are diluted (doubling dilutions) in 2% FBS in RPMI 1640 up to 11 wells with the twelth well containing diluent only as a control. 25 ul of diluent plus MAb (at a concentration of 2 MHD) is added to each well. 30 uL of the RBC suspension is added to each well and left to settle at room temperature or in the fridge overnight. If the MAb binds the test antigen, then it is no longer available to agglutinate the RBCs and the RBCs will settle into a button on the base of the well. As the antigen becomes increasingly dilute, more of the MAb remains free to agglutinate the RBCs which should be obvious in the control well without antigen.

The assay detects FLC both as dimer and monomer free in solution.

Results: MAb dilution 1/100,000 vs BJ kappa Bo

| | | |
|---|---|---|
| IgG1 kappa Bow | 5 | free kappa in the preparation |
| IgG1 kappa Re | 0 | |
| IgG1 kappa Cr | 4 | |
| IgG2 kappa Mi | 0 | |
| IgG2 kappa Ca | 1 | |
| IgG2 lambda M3 | 0 | |
| IgG3 kappa Pi | 0 | |
| IgG3 kappa Gi | 0 | |
| IgG4 kappa Rei | 5 | |
| IgG4 kappa Jon | 0 | |
| BJ kappa Bo | 11 | |
| BJ kappa Ne | 9 | |
| BJ kappa Na | 11 | |
| BJ kappa Ke | 12 | |
| BJ kappa Ho | 9 | |
| BJ kappa Re | 10 | |
| BJ lambda Ev | 0 | |
| Polyclonal IgG | 0 | |
| Fab | 4 | |
| F(ab)2 | 8 | |
| Fab Bow IgG1 | 10 | |
| F(ab)2 Bow IgG1 | 9 | |
| F(ab)2 Pi IgG3 | 4 | |
| Fab Gi | 4 | |

Production:

Cells were accessed from storage in liquid nitrogen and grown in RPMI 1640 containing penicillin (120 ug/ml) and streptomycin (100 units/ml) and supplemented with 10% fetal bovine serum. Productivity and specificity was confirmed by ELISA and HA. Cells were bulked up for loading a MiniPerm bioreactor (Greiner Bio-One) and loaded at 5×10⁷ cells. The medium in the reservoir was RPMI 1640 with penicillin and streptomycin and 1 g/L extra glucose. The production module medium was the same supplemented with 10% fetal bovine serum which had been depleted of most of the bovine IgG by passing down an SpA column. Once established, the bioreactor was harvested every 2 days by removing the cell suspension with a syringe. The cells were spun down (1500 rpm for 3 mins), the supernatant decanted and the cells resuspended in fresh medium and returned to the production module. The supernatant was passed through a 0.2 um filter before storing at −20° C. or purifying.

Purification:

(see SOPs for buffer recipes for protein purifications and chromatography methodologies)

Protein G coupled to Sepharose 4 fast flow—GE Healthcare 17-0618-01 5 ml

Equilibration/wash buffer—PBS pH 7.2 (sodium salts)

Elution buffer—0.1 M citrate pH 2.4

Neutralisation buffer—1M citrate phosphate buffer pH 8.

Column storage buffer—PBS/Azide (0.1%)

Monoclonal antibody presented in culture medium as cell supernatant:

Check the pH and, if necessary, adjust to pH 7.2 with 1M HCl or 1M NaOH as appropriate. 0.2 u filter. Estimate the concentration of MAb in the supernatant (based on assay titre and previous purifications). Pipette the required volume of supernatant into a sterile container.

Wash the column with PBS, at least 3 bed volumes, and establish base line. Load column with supernatant and collect last third of the volume loaded in case of overload and to monitor the efficiency of retrieval of the MAb (ELISA or HA assay).

Wash with PBS and collect washings as some leaching may take place especially if the column is nearing its capacity.

Elute with acid: collect in glass tubes starting with 50 ul neutralisation buffer in tubes. Begin collection when chart recorder indicates concentration of eluate is approaching 0.2 mg/ml. To 300 ul of eluate add 50 ul neutralising buffer. Continue in like manner collecting in series of tubes (approx.5 ml per tube) as required. Mix. Continue until concentration falls to below 0.5 mg/ml. and collect “tail” in a separate tube. Quickly check the pH of each tube and add neutralising buffer to achieve a pH of at least 6.5 but no more than 6.8.

When base line is reached, wash with PBS for another run or wash with PBS/azide for storage of the column at 2-8° C.

Check breakthrough and washings for MAb content (see SOPs for antibody assays). Pass down column again if worthwhile.

Determine the concentration of protein in each tube by spectrophotometry at 280 A and a coefficient of extinction of 14.3.

Pool appropriate tubes to give a concentration of at least 1 mg/ml

Dialyse eluate against PBS (50 to 100 volumes of dialysis buffer) for at least 4 hrs on a magnetic stirrer and/or overnight at 2-8 C. (see SOP for dialysis)

Filter sterilise (0.2 u) (see SOP) purified MAb and take 50 ul for concentration determination by spectrophotometer (see SOP for protein concentration determination) and purity assays (ELISA, SDS PAGE etc). Minimum concentration is 1 mg/ml.

Aliquot 0.5 ml for quality control. Use to “coat” beads for Luminex assay and determine the efficiency of coating. Determine the binding of biotinylated FLC to the coated beads and obtain a standard curve.

J—Anti Lambda FLC Monoclonal Antibody 21C3 (LC3)—Details and Preparation

Murine monoclonal antibody (MAb) specific for human free lambda light chains i.e. NOT bound to the heavy chain of immunoglobulins of any class. Raised by fusion of mouse balb/c spleen cell with 653 immortal partner.

Immunogen: human lambda free light chain preparation from urine containing Bence Jones protein.

Murine IgG class; IgG1 isotype.

Specificity determined by Heamagglutination (HA) and Haemagglutination Inhibition (HAI) of sheep red blood cells coated with purified human serum proteins. (see Ling, Bishop and Jefferis 1977 J. Immunol. Methods. 15:279)

Human IgG, IgA, IgM, IgD and FLC preparations used to confirm the specificity. Also confirmed using an ELISA (Enzyme Linked ImmunoSorbent Assay).

Original Specificity Determination:

Haemagglutination Inhibition (HAI) (Ling, Bishop and Jefferis {1977} J Imm. Methods 15: 279-289. Use of antibody-coated red cells for the sensitive detection of antigen and in rosette tests for cells bearing surface immunoglobulins).

Sheep RBCs are covalently linked with BJ lambda protein Ev using Chromium chloride. The monoclonal antibody is titrated (25 uL volumes in a 96 round bottomed well plate) against these coated RBCs to determine the concentration of MAb which will still give obvious agglutination. This is the minimum haemolytic dose (MHD). A stock of antibody at a concentration of 2MHD in diluent (2% FBS in RPMI 1640 medium) is made up. The antigens ie human serum immunoglobulins, are diluted (doubling dilutions) in this MAb-containing-diluent up to 11 wells with the twelfth well containing MAb-diluent as a control. 30 uL of the RBC suspension is added to each well and left to settle at room temperature or in the fridge overnight. If the MAb binds the test antigen, then it is no longer available to agglutinate the RBCs and the RBCs will settle into a button on the base of the well. As the antigen becomes increasingly dilute, more of the MAb remains free to agglutinate the RBCs which should be obvious in the control well without antigen.

Results: MAb dilution 1/100,000 vs BJ lambda Ev

| | | |
|---|---|---|
| IgG1 lambda Sm | 0 | |
| IgG1 lambda Gr | 2 | some free lambda in the preparation |
| IgG1 kappa Re | 0 | |
| IgG kappa Ha | 0 | |
| IgG2 lambda Gi | 0 | |
| IgG2 lambda Hi | 0 | |
| IgG2 lambda Su | 0 | |
| IgG3 lambda Wil | 1 | |
| IgG3 lambda Ra | 0 | |
| IgG3 lambda Re | 0 | |
| IgG4 lambda Ca | 0 | |
| IgG4 lambda Wis | 5 | some free lambda in the preparation |
| BJ lambda Ev | 12 | |
| BJ lambda We | 5 | |
| BJ lambda Ke | 13 | |
| BJ lambda Hu | 4 | |
| BJ lambda Sn | 5 | |
| BJ kappa Bo | 1 | |
| Fab | 0 | |
| F(ab)2 | 0 | |
| Fc | 0 | |

Production:

Cells were accessed from storage in liquid nitrogen and grown in RPMI 1640 containing penicillin (120 ug/ml) and streptomycin (100 units/ml) and supplemented with 10% fetal bovine serum. Productivity and specificity was confirmed by ELISA and HA. Cells were bulked up for loading a MiniPerm bioreactor (Greiner Bio-One) and loaded at 5×10⁷ cells. The medium in the reservoir was RPMI 1640 with penicillin and streptomycin and 1 g/L extra glucose. The production module medium was the same supplemented with 10% fetal bovine serum which had been depleted of most of the bovine IgG by passing down an SpA column. Once established, the bioreactor was harvested every 2 days by removing the cell suspension with a syringe. The cells were spun down (1500 rpm for 3 mins), the supernatant decanted and the cells resuspended in fresh medium and returned to the production module. The supernatant was passed through a 0.2 um filter before storing at −20° C. or purifying.

Purification:

(see SOPs for buffer recipes for protein purifications and chromatography methodologies)

Protein G coupled to Sepharose 4 fast flow—GE Healthcare 17-0618-01 5 ml

Equilibration/wash buffer—PBS pH 7.2 (sodium salts)

Elution buffer—0.1M citrate pH 2.4-2.6

Neutralisation buffer—1M citrate/phosphate pH 8.0—MUST BE KEPT WARM in water bath or crystallises because close to saturation point.

Column storage buffer—PBS/Azide (0.1%)

Monoclonal antibody presented in culture medium as cell supernatant:

Check the pH and, if necessary, adjust to pH 7.2 with 1M HCl or 1M NaOH as appropriate. 0.2 u filter. Estimate the concentration of MAb in the supernatant (based on assay titre and previous purifications). Pipette the required volume of supernatant into a sterile container.

Wash the column with PBS, at least 3 bed volumes, and establish base line.

Load column with supernatant and collect last third of the volume loaded in case of overload and to monitor the efficiency of retrieval of the MAb (ELISA or HA assay). Wash with PBS and collect washings as some leaching may take place especially if the column is nearing its capacity.

Elute with acid: collect in glass tubes starting with 50 ul neutralisation buffer in tubes. Begin collection when chart recorder indicates concentration of eluate is approaching 0.2 mg/ml. To 300 ul of eluate add 50 ul neutralising buffer. Continue in like manner collecting in series of tubes (approx. 5 ml per tube) as required. Mix. Continue until concentration falls to below 0.5 mg/ml. and collect “tail” in a separate tube. Quickly check the pH of each tube and add neutralising buffer to achieve a pH of at least 6.5 but no more than 6.8.

When base line is reached, wash with PBS for another run or wash with PBS/azide for storage of the column at 2-8° C.

Check breakthrough and washings for MAb content (see SOPs for antibody assays). Pass down column again if worthwhile.

Measure protein concentration of each tube by spectrophotometry at 280 A and using a coefficient of extinction of 14.3 for IgG (Hay and Westwood)

Pool appropriate tubes to give a concentration of more than 1 mg/ml.

Dialyse eluate against PBS (50 to 100 volumes of dialysis buffer) for at least 4 hrs on a magnetic stirrer and/or overnight at 2-8 C. (see SOP for dialysis)

Filter sterilise (0.2 u) (see SOP) purified MAb and take 50 ul for concentration determination by spectrophotometer (see SOP for protein concentration determination) and purity assays (ELISA, SDS PAGE etc). Minimum concentration is 1 mg/ml.

Aliquot 0.5 ml for quality control. Use to determine the efficiency of “coating” beads for Luminex assay, the binding of biotinylated FLC to the coated beads and production of the standard curve.

### EXAMPLE 2

### A Method of Measuring the Ratio of Free κ Immunoglobulin Light Chain Molecules to Free λ Immunoglobulin Light Chain Molecules in a Test Sample

The following example provides detailed instructions for performing an embodiment of the method of the invention. The reagents and methods used below are further described above in Example 1.

This particular embodiment is an “inhibition assay”, in which the measurement of the amount of free κ light chain to free λ light chain in a test sample is calculated according to the reduction of binding of a fixed concentration of labelled κ and λ light chain molecules to microspheres.

Luminex microspheres are manufactured with variable levels of two proprietary fluorescent dyes such that 100 types are available termed ‘regions’. Each microsphere can be distinguished from each other by measuring the fluorescence of the two dyes. Two types of Luminex microspheres are used in this method and are coated with either monoclonal antibody that can bind to free κ made by hybridoma cell line KA1 or monoclonal antibody that can bind to free λ made by hybridoma cell line 21C3.

Alternatively, encoded aluminium bars supplied by Pronostics can be used as support material having a coating of monoclonal antibody can be used in the assay method.

1. Preparation of the Luminex Microspheres.

Coating of the monoclonal antibody as defined in the specification onto the Luminex microspheres is performed as set out in the supplier's instructions, and as set out above. Luminex microspheres are tested for the coating-efficiency and the reproducibility of the mAb preparation (using the standard curve protein preparations); protocols for both methods are set out above.

2. Biotinylation of Labelled Free Light Chains.

The method of this example uses labelled free light chains molecules that are purified from urine of patients excreting high concentrations of the light chains (a protocol for preparing free light chains molecules is provided in example 1 section A above). They are conjugated to biotin as follows:

40 uL of 1M sodium bicarbonate buffer (pH 8.3-9.0) is added to 400 uL of each of the two free light chain preparations κ and λ.

Biotin NHS (Biotinamidohexanoyl 1-6-aminohexanoic acid N-hydroxysuccinimde ester. Minimum 95%) is dissolved in DMSO (dimethyl sulfoxide minimum 99.5%) at 10 mg/mL 200 uL DMSO to 2000 ug Biotin NHS slowly while stirring on a rotary mixer. Incubate 1 hr at room temperature on rotary mixer. Buffer exchange into PBS (phosphate buffered saline) is performed using NAP™ columns (GE Healthcare) dedicating a column for each light chain type. The efficiency of biotinylation is tested and compared with previous batches. The biotinylated protein can be stored with the addition of 0.09% azide as preservative when the shelf life can be as long a 3 months but the kappa and lambda preparations must be stored separately and mixed just before use.

3. Preparation of Free Light Chain Protein for Standard Curve.

The same proteins used for biotinylation are used for a standard curve against which the concentration of light chain in the patient's sera can be quantified. A mixture of light chain protein from at least 4 sources (in equal amounts) is prepared at 7 mg/ml.

A 1 in 4 dilution range of concentrations is used from 1750 μg/ml (mg/L) down to 0.11 μg/ml (mg/L).

4. Assay Procedure

Biotinylated FLC is mixed with the assay reagent containing the monoclonal antibodies bound to the Luminex microsphere support material and then incubated with streptavidin R-phycoerythrin conjugate (SA-PE) from Molecular Probes. After washing, the microspheres are analysed on the Luminex machine. The streptavidin specifically binds to the biotin on the FLC molecules and the R-phycoerythrin fluoresces light at its own specific wavelength in the red region of the spectrum. The machine measures the intensity of the red light refracted by each of 50 microspheres (the intensity from any one microsphere will be proportional to the amount of biotinylated FLC bound to the mAb on the microsphere). The machine computes the mean intensity and relates it to the code incorporated into the microspheres.

Without the presence of any non conjugated FLC, the result will give the maximum intensity of binding of the labelled molecules to the microsphere. When non-conjugated FLC is added into the reaction, either from the standard curve samples, or from a patient test sample, there is competition for the MAb on the surface of the microsphere: the more non-conjugated FLC present, the less conjugated FLC will be bound and thus the mean intensity will be less and the signal reduced. The results can be computed to be compared with the standard curve to give the concentrations of FLC in the patient test sample. (The results are inverted because it is an inhibition assay).

Multiple different microspheres species each coupled with a different mAb, can be combined in the same well so that several different assays e.g. κ-FLC, λ-FLC, can be assayed simultaneously on the same sample and each microsphere identified using the “region”.

### EXAMPLE 3

### Measurement of Serum Immunoglobulin Free Light Chains

Introduction

Laboratory detection of M-proteins for diagnostic purposes centres on the detection of whole immunoglobulin M-protein in the serum and of free light chain (FLC) immunoglobulin M-protein (Bence Jones Protein) in the urine. Only one laboratory kit for measurement of FLC has been able to detect the few mg/L of FLC present in serum without interference from the g/L of light chain bound into whole immunoglobulin. This nephelometric assay for serum FLC (Freelite™) uses polyclonal sheep sera (Bradwell, A. R. et al. Highly sensitive, automated immunoassay for immunoglobulin free light chains in serum and urine. Clin. Chem. 47, 673-680 2001). This free light chain assay is specific for free κ and λ light chains and does not recognize light chains bound to intact immunoglobulin.

Using quantitative nephelometry, Freelite™ measures the serum κ and λ free light chain levels in separate assays which also allows calculation of the free light chain κ:λ ratio.

The inventors have developed an alternative assay for serum FLC using murine monoclonals specific for free light chains in a multiplexed system. The binding molecules used in the improved method have much improved consistency and sensitivity over the polyclonal antibodies used in the existing assays, and do not rely on an animal system for production. Furthermore the improved method of the invention can detect both κ and λ free light chain types simultaneously; the method can measure light chains in urine and serum equally effectively; and it provides a four decade range of sensitivity and is much faster and less wasteful than the existing assay.

Methods and Results

Monoclonal Antibodies (mAb)

The inventors have undertaken a systematic screen of monoclonal antibodies (mAb) to identify those which can recognise human serum free kappa (sfκ) and serum free lambda (sfλ). They have investigated over 20 mAbs under a number of different assay techniques with the aim of identifying mAbs which can accurately measure these analytes in a multiplexed microsphere array based assay. They identified two mAbs which perform exceptionally well for this purpose: hybridoma cell line KA1 produces a mAb that specifically binds to free κ immunoglobulin light chain molecules; hybridoma cell line 21C3 produces a mAb that specifically binds to free λ immunoglobulin light chain molecules. These assays perform well for both urine and serum patient samples with high sensitivity and specificity. Binding characteristics for the KA1 and 21C3 MAbs are shown in accompanying Examples 4 and 5.

Validation

To validate the assay the inventors determined the sfκ and sfλ levels in 600 patients with myeloma or related diseases and 200 serum samples from healthy individuals. The inventors used the assay method outlined in Example 2, in which the mAbs were bound to Luminex microspheres. Samples were analysed in parallel using Luminex based assay and the Freelite™ assay. Results are shown in FIG. 1. It is important to point out that the data presented represents a single multiplexed Luminex assay of a 1:100 serum dilution, whereas the Freelite™ value represents results from multiple dilutions of patient sera.

FIG. 2 then shows a comparison of Freelite™ KLR vs Luminex KLR. It includes an ROC curve of Luminex Assay comparing 200 normal with 600 patient sera (Showing correlation of 600 patient sera of Freelite™ with the inevntors assay). This shows the distribution of 200 normal sera samples (blue) with 600 patient samples (red) demonstrating distinct populations. Top=Luminex Assay; Bottom=Freelite™

FIG. 3 then shows a comparison of the measurement of urine free κ and λ by a flow cytometric (Luminex) based assay. (A) the correlation of urine free λ as measured by radial immunodiffusion (RID) using the 21C3 and 21E9 monoclonal antibodies. (B) the correlation of urine free κ as measured by RID using the KA1 and B3B4 monoclonal antibodies.

### SUMMARY

The table below sets out the improvements offered by the assay method devised by the inventors.

TABLE 1

| | Inventor Assay | Freelite |
|---|---|---|
| Immunoassay design | Murine monoclonal | Sheep polyclonal |
| | Microsphere Array | Latex enhanced |
| | | nephelometry |
| Limit of detection | 0.01 mg/L | 0.8 mg/L (Neat serum) |
| Dynamic Range^{†} | 1-1,000 mg/L | 4-56 mg/L (1:5 dilution) |
| Limitations | | |
| Antigen Excess | No | Yes |
| Multiplexed | Yes | No |
| Standard | No | Yes |
| instruments* | | |

*Can run on standard clinical instruments (eg Roche Hitachi; Dade Behring; Olympus; Beckman)
^{†}Required detection range of clinical samples 0.5-100,000 mg/L

Measuring low levels is crucial because it determines the degree of immunoparesis in the alternate polyclonal light chains which is fundamental to the clinical sensitivity of the assay.

### EXAMPLE 4

### Sandwich and Competitive Inhibition ELISA

A. Background

To investigate whether KA1 and 21C3 monoclonal antibodies could be used in a capture-based system, κ and λ ELISA (Enzyme Linked ImmunoSorbent Assay) plates were set up and tested. The protocol set out below can also be used to determine the binding characteristics of other MAbs to κ or λ.

This involved coating anti-κ and anti-λ free light chain antibodies onto ELISA plates, then adding antigens and clinical samples which either should or should not bind to the antibody. Next, an enzyme-linked secondary antibody (conjugated to peroxidase) specific for each target-antigen and a substrate catalysed by peroxidase to yield a coloured product are applied to the plates. This enables the relative amounts of antigen bound to its antibody to be quantified by reading the absorbance of each well at 450 nm.

B. Buffers and Preservatives

1. Phosphate Buffered Saline (PBS).

Dissolve 10 phosphate buffered saline Dulbecco A tablet (Oxoid, UK BR0014G) in 1000 ml distilled water. Check is pH 7.4+/−0.2 on a pre-calibrated pH meter.

2. PBS-Tween

Take 1 L PBS (as prepared in section 1) and add 1 ml Tween 20 (polyoxyethylene sorbitan monolaurate; Sigma P1379).

3. Blocking Solution

To 100 ml of PBS (prepared as in section 1) and add 2 g Bovine Serum Albumin (BSA; Sigma A4503). Gently rock to dissolve BSA.

4. TMB

3,3′,5,5′-Tetramethylbenzidine (TMB) Liquid Substrate System for ELISA (Sigma T0440)

C. Kappa and Lambda FLC Competitive ELISA Assay

Standard curve consists of 8 points: 1750 mg/L; 440 mg/L; 110 mg/L; 27 mg/L; 6.8 mg/L; 1.7 mg/L; 0.4 mg/L; 0.1 mg/L.

Day 0

1. One κ and one λ Maxisorp™ ELISA plates (Nunc) plates were set up, coating the KA1 and 21C3 mAbs respectively. Antibodies were diluted to 10 μg/ml in PBS and 50 μl added to each well. Each plate is coated with EITHER KA1 or 21C3 not both. Incubate the plates in a humid container within a refrigerator overnight.

Day 1 Competitive ELISA

2. Prepare a standard curve.

Dilute κ-FLC and λ-FLC standard curve protein from 7 mg/ml stock in 4 fold dilution curve such that first standard has κ-SFL at 1750 mg/L and λ-SFL at 1750 mg/L (i.e. Mix 30 μl κ-SFL at 7 mg/ml+30 μl λ-SFL at 7 mg/ml+60 μl dilution buffer to make 120 μl of upper standard−1750 mg/L. Take 30 μl of this and add to 90 μl of dilution buffer to generate serial dilutions from 440 mg/L; 110 mg/L; 27 mg/L; 6.8 mg/L; 1.7 mg/L; 0.4 mg/L down to 0.1 mg/L) to give 90 μl of each dilution. It is recommended that the standard curve is run in duplicate and thus a total of 2×40 μl=80 μl of each standard will be needed.

3. Biotinylated κ and λ light chains are prepared in the same way as Example 1 section A

4. The PBS-mAb is removed from each well. Each well is washed 3 times with 200 ul of PBS-Tween.

5. 200 μl of blocking solution is added to each well and the plate incubated at room temperature for 1 hour in the dark.

6. After incubation with the blocking solution the plates were washed with 200 μl of PBS-Tween wash buffer three times and once with PBS.

7. 50 ul of standards (from step 2 above) are then added to each well and samples added to other wells.

8. 50 μl of either biotinylated κ-flc or biotinylated λ-flc diluted 1:1000 with PBS is added to each well.

9. Plate is incubated at room temperature for 2 hours with shaking.

10. Fluid is decanted from each well on the plate each well of which is washed 6 times with 200 μl PBS-Tween

11. A solution of Streptavidin-HRP (Sigma) was made up at 1 μg/ml in PBS and 100 μl added to each well and incubated at room temperature for 30 minutes.

12. Plate is washed 6 times with 200 μl PBS-Tween, then 200 μl of PBS

13. All fluid is discarded and 100 μl of TMB solution is added to each well.

14. Plate is incubated for 10 minutes.

15. 100 μl of stop solution (1M orthophosphoric acid) is added to each well without removing the TMB.

16. Plate is read using spectrophotometer plate-reader at 405 nm and 450 nm.

17. A standard curve is generated and concentration of either κ or λ flc determined.

D. Kappa and Lambda FLC Sandwich ELISA Assay

Standard curve consists of 8 points: 1750 mg/L; 440 mg/L; 110 mg/L; 27 mg/L; 6.8 mg/L; 1.7 mg/L; 0.4 mg/L; 0.1 mg/L.

Day 0

1. One κ and one λ Maxisorp™ ELISA plates (Nunc) plates were set up, coating the KA1 and 21C3 mAbs respectively. Antibodies were diluted to 10 μg/ml in PBS and 50 μl added to each well. Each plate is coated with EITHER KA1 or 21C3 not both. Incubate the plates in a humid container within a refrigerator overnight.

2. Biotinylated anti-kappa (free and bound) and Biotinylated anti-λ (free and bound) are either sources commercially or prepared. In this assay we have used our in-house mAb 312H (anti-λ free and bound) and 6EL (anti-κ heavy and bound). These antibodies were biotinylated using the method described in example 1 section A above.

Day 1 Sandwich ELISA

2. Prepare a standard curve.

Dilute κ-FLC and λ-FLC standard curve protein from 7 mg/ml stock in 4 fold dilution curve such that first standard has κ-SFL at 1750 mg/L and λ-SFL at 1750 mg/L (i.e. Mix 30 μl κ-SFL at 7 mg/ml+30 μl λ-SFL at 7 mg/ml+60 μl dilution buffer to make 120 μl of upper standard−1750 mg/L. Take 30 μl of this and add to 90 μl of dilution buffer to generate serial dilutions from 440 mg/L; 110 mg/L; 27 mg/L; 6.8 mg/L; 1.7 mg/L; 0.4 mg/L down to 0.1 mg/L) to give 90 μl of each dilution. It is recommended that the standard curve is run in duplicate and thus a total of 2×40 μl=80 μl of each standard will be needed.

3. The PBS-mAb is removed from each well. Each well is washed 3 times with 200 ul of PBS-Tween.

4. 200 μl of blocking solution is added to each well and the plate incubated at room temperature for 1 hour in the dark.

5. After incubation with the blocking solution the plates were washed with 200 μl of PBS-Tween wash buffer three times and once with PBS.

6. 50 ul of standards (from 2) are then added to appropriate wells and samples added to other wells.

7. Plate is incubated at room temperature for 2 hours with shaking.

8. Fluid is decanted from each well on the plate each well of which is washed 6 times with 200 μl PBS-Tween

9. A biotinylated mAbs (prepared in step 1) are diluted in blocking buffer at 0.1 μg/ml and 50 μl is then added to each well. Biotinylated 312H is used for detecting λ (pairing with 21C3 or 21E9) and 6EL is used for detecting κ (pairing with KA1 or B3B4).

7. Plate is incubated at room temperature for 1 hour with shaking.

8. Fluid is decanted from each well on the plate each well of which is washed 6 times with 200 μl PBS-Tween

11. A solution of Streptavidin-HRP (Sigma) was made up at 1 μg/ml in PBS and 100 μl added to each well and incubated at room temperature for 30 minutes.

12. Plate is washed 6 times with 200 μl PBS-Tween, then 200 μl of PBS

13. All fluid is discarded and 100 μl of TMB solution is added to each well.

14. Plate is incubated for 10 minutes.

15. 100 μl of stop solution (1M orthophosphoric acid) is added to each well without removing the TMB.

16. Plate is read using spectrophotometer plate-reader at 405 nm and 450 nm.

17. A standard curve is generated and concentration of either κ or λ flc determined.

E—Results

The absorbance results obtained for Kappa and Lambda demonstrate that both of the KA1 and 21C3 mAbs are able to work effectively in an ELISA-based antigen capture system. FIG. 4 shows kappa and lambda ELISAs for a competitive inhibition assay and FIG. 5 shows the results for a sandwich ELISA assay. FIG. 5 demonstrates that combining additional monoclonal antibodies (to make a pair) allows detection of serum free light chains without the requirement for the competitor (biotinylated lightchain).

None of the control wells had absorbances significantly higher than the substrate blank for each plate, so no significant absorbance can be attributed to background from any of the antigens/samples or secondary antibodies.

There is no cross-reaction with IgGλ or IgGκ heavy chain.

The skilled person will understand that alternative detection methods, to detect κ or λ free light chains bound to their respective antibodies, may be used. For example, the secondary antibodies could be labelled and detected using a system such as that outlined in. In this disclosure, molecules are labelled with, for example, isothiocyanate derivatives, for example a derivative of Tb(IH)-I chelate. Molecules with such derivatives bound can be detected by causing the chelate to enter an excited state by applying electrical pulses; as the chelate spontaneously returns to the non-excited state, ultraviolet, visible or infrared light is emitted. This light can be detected and a quantitative measure of the amount of κ or λ free light chains bound to their respective antibodies obtained.

Alternatively, the secondary antibody can be labelled with a fluorescent probe, which fluoresces on exposure to, for example, ultraviolet light. Once the sample had been exposed to the primary and secondary antibodies, the sample is further exposed to ultraviolet light. The resulting fluorescence is detected and a quantitative measure of the amount of κ or λ free light chains bound to their respective antibodies obtained.

### EXAMPLE 5

### Agglutination Inhibition Assay

A: Introduction

This section describes to utility of the KA1 and 21C3 mAbs to detect free light chains using an agglutination inhibition assay. In this system red blood cells are labelled with either free-κ or free-λ. The mAb KA1 or 21C3 are then added which will cause agglutination of the coated red cells. The minimal concentration of either KA1 or 21C3 which will induce this is next determined—termed minimal haemagglutinating dose (MHD). For the assay, the 21C3 or KA1 (at the predefined concentration of MHD) are added to serial doubling dilutions of test-sera. Free light chains within the test sera will bind to the 21C3 or KA1 and thus inhibit the agglutination. The dilution (titre) of sera where agglutination is not inhibited is used to calculate the concentration of light chain in the sample against a standard curve. The assay set out below can also be used to determine the binding characteristics of other MAbs to κ or λ.

B-Methods

The method is based on methodology first published (Ling, Bishop and Jefferis, J Imm. Methods 15: 279-289. 1977. Use of antibody-coated red cells for the sensitive detection of antigen and in rosette tests for cells bearing surface immunoglobulins)

1. Sheep red blood cells were coated with free kappa light chains and free lambda light chains respectively.

2. The purified mAbs were titrated in doubling dilutions in RPMI+2% FCS and the end point determined at which agglutination was observed. This minimal concentration at which haemagglutination was observed was termed the minimal haemagglutinating dose (MHD).

3. Stock solution of mAb at a concentration of twice MHD (2×MHD) was made up and checked for Haemagglutination.

4. The test sera were first diluted 1:10 and subsequently doubly diluted in 25 μl volumes of RPMI+2% FCS.

5. To each well 25 μl of diluent containing 2×MHD of mAb and then to each well 25 ul of coated sheep red blood cell suspension.

6. Plates are left for 1 hour at room temperature for the red cells to settle.

7. Agglutination was determined via macroscopic visualisation and the titre was determined where agglutination first observed (ie where agglutination was not inhibited).

8. Patient sera were analysed in parallel with known standards and the concentration was determined and plotted against results as obtained via the FREELITE™ assay.

C: Results

FIG. 6 shows the correlation between κ-SFL and λ-SFL analysis of 14 serum samples as determined by Freelite™ and the haemagluttination inhibition assay. A very good correlation was found showing that this method accurately measures both κ and λ serum free light chains in the sera, but not in the same assay. This assay has a sensitivity of around 1 mg/L of both κ and λ with a correlation coefficient of 0.85 and 0.03 respectively.

It is anticipated that these mAbs will be suitable for any bead enhanced turbidimetric or nephelometry based assay (particle-enhanced turbidimetric inhibition assay; particle-enhanced nephelometric inhibition assay) would work as the principles of these assays are the same as the haemagglutination.

### EXAMPLE 6

### Amino Acid Sequence of the Hypervariable Region from Monoclonal Antibodies 1B6, 21C3 and KA1

Using standard molecular biology techniques, the inventors isolated then sequenced the amino acids from the hyperviariable region of 1B6, 21C3 and KA1. Annotated amino acid sequence is provided below.

A. Monoclonal Antibody 1B6—Free Kappa Antibody

1B6 Ig Heavy Chain Region

IgH V3-2*02 (97.75% homology):
RQFPGNKLEWMGYINYSGITSYNPSLKSRFSITRDTSKNQFFLQLNS
VTTEDTATYY
CDR3 Region:
CASWYYGNWYFDVW
D-J Region:
GAGTTVTV
Constant Region:
SSAKTTPPSVYPL

The amino acid sequence of the hypervariable region of the heavy chain of 1B6 therefore comprises:

RQFPGNKLEWMGYINYSGITSYNPSLKSRFSITRDTSKNQFFLQLNSVT
TEDTATYY CASWYYGNWYFDVW

This corresponds to the CDRs 1, 2 and 3 and framework regions.

1B6 Kappa Chain

IgK Variable 12-46*01:
DIQMTQSPASLSVSVGETVTITCRASENIYSNLAWYQQKQGNSPQLLV
YAATNLADGVPSRFSGSGSGTQYSLKINSLQSEDFGNYY
CDR3:
CQHFWGTPWTF
IgK Joining Region:
GGGTKLEIKR
Constant Region:
ADAAPTVSIFPPSSKGEF

The amino acid sequence of the hypervariable region of the kappa chain of 1B6 therefore comprises:

DIQMTQSPASLSVSVGETVTITCRASENIYSNLAWYQQKQGNSPQLLVY
AATNLADGVPSRFSGSGSGTQYSLKINSLQSEDFGNYYCQHFWGTPWTF

This corresponds to the CDRs 1, 2 and 3 and framework regions.

B. Monoclonal Antibody 21C3—Free Lambda Antibody

21C3 Ig Heavy Chain Region

IgH V2-9-1*01 (96.5% homology):
VRQPPGKGLEWLGVLWAGGSTNYNSALMSRLSIHKDNSKSQFFL
KMNSLQADDTAMYY
CDR3 Region:
CARARSAMDYW
D-J Region:
GQGTSVTV
Constant Region:
SSAKTTPPSVYPLKGEF

The amino acid sequence of the hypervariable region of the heavy chain of 21C3 therefore comprises:

VRQPPGKGLEWLGVLWAGGSTNYNSALMSRLSIHKDNSKSQFFLKMN
SLQADDTAMYYCARARSAMDYW

This corresponds to the CDRs 1, 2 and 3 and framework regions.

21C3 Kappa Chain

IgK Variable 3-4*01:
DIVLTQSPASLAVSLGQRATISCKASQSVDYDDNSYMNWYQQKPGQPP
KLLIYAASNLESGIPARFSGSGSGTDFTLNIHPVEEEDAATYY
CDR3:
CQQSNED
IgK Joining Region:
PYTFGGGTKLEIKR
Constant Region:
ADAAPTVSIF

The amino acid sequence of the hypervariable region of the kappa chain of 21C3 therefore comprises:

DIVLTQSPASLAVSLGQRATISCKASQSVDYDDNSYMNWYQQKPGQPPK
LLIYAASNLESGIPARFSGSGSGTDFTLNIHPVEEEDAATYYCQQSNED

This corresponds to the CDRs 1, 2 and 3 and framework regions.

C. Monoclonal Antibody KA1—Free Kappa Antibody

KA1 Ig Heavy Chain Region

IgK Variable 3-2*02:
DVQLQESGPGLVNPSQSLSLTCTVTGYSITGDYAWNWIRQFPGNKLEW
MGYISYSGLTSYNPSLKSRISITRDTSKNQFFLQLNSVATEDTATYY
CDR3:
CTSFYYGYWYFD
IgK Joining Region:
VWGAGTTVTV
Constant Region:
SSAKTTPPSVYPLKGEF

The amino acid sequence of the hypervariable region of the heavy chain of KA1 therefore comprises:

DVQLQESGPGLVNPSQSLSLTCTVTGYSITGDYAWNWIRQFPGNKLEW
MGYISYSGLTSYNPSLKSRISITRDTSKNQFFLQLNSVATEDTATYYC
TSFYYGYWYFD

This corresponds to the CDRs 1, 2 and 3 and framework regions.

KA1 Kappa Chain

IgK Variable 12-44*01:
DIQMTQSPASLSASVGETVTITCRASENIYSFLTWYLQKQGKSPQLLV
NNAKTLAEGVPSRFSGSGSGSQFSLKINSLQPEDFGTYY
CDR3:
CQHHYGTPYTF
IgK Joining Region:
GGGTKLEIKRAD
Constant Region:
AAPTVSIFPPSSK

The amino acid sequence of the hypervariable region of the kappa chain of KA1 therefore comprises:

DIQMTQSPASLSASVGETVTITCRASENIYSFLTWYLQKQGKSPQLLVN
NAKTLAEGVPSRFSGSGSGSQFSLKINSLQPEDFGTYYCQHHYGTPYTF

This corresponds to the CDRs 1, 2 and 3 and framework regions.

### EXAMPLE 7

### Competitive Inhibition Assays to Measure Levels of Free Light Chain in Urine

We used the anti-free kappa and anti-free lambda monoclonal antibodies of the invention and described herein in competitive inhibition assays to measure levels of free light chain in urine. Using the luminex bead array platform and assay method described above, we measured free light chain levels in 6881 samples (data not shown). The results concurred with other laboratory tests for plasma cell dyscrasias in the same samples.

This experiment demonstrates that the anti-free kappa and anti-free lambda monoclonal antibodies of the invention can be used to detect monoclonal free light chains from a very wide range of patients. This demonstrates the utility of the antibodies and methods of the invention.

1. A method of measuring the ratio of free κ immunoglobulin light chain molecules to free λ immunoglobulin light chain molecules in a test sample comprising:
(i) measuring the amount of free κ in the test sample using a monoclonal antibody, or an antigen binding fragment or variant of said monoclonal antibody, that specifically binds to free κ;
(ii) measuring the amount of free λ in the test sample using a monoclonal antibody, or an antigen binding fragment or variant of said monoclonal antibody, that specifically binds to free λ;
(iii) calculating the ratio of free κ to free λ levels in the test sample.

2. The method of claim 1 wherein the variable region of the heavy chain of the monoclonal antibody that specifically binds to free κ comprises the amino acid sequence provided in SEQ ID NO:1, and/or the variable region of the light chain of the monoclonal antibody that specifically binds to free κ comprises the amino acid sequence provided in SEQ ID NO:2.

3. The method of claim 2 wherein the monoclonal antibody that specifically binds to free κ is produced by the hybridoma cell line KA1, deposited under Budapest Treaty at the ECACC under reference number 08071702.

4. The method of claim 1 wherein the variable region of the heavy chain of the monoclonal antibody that specifically binds to free κ comprises the amino acid sequence provided in SEQ ID NO:3, and/or the variable region of the light chain of the monoclonal antibody that specifically binds to free κ comprises the amino acid sequence provided in SEQ ID NO:4.

5. The method of claim 4 wherein the monoclonal antibody that specifically binds to free κ is produced by the hybridoma cell line 1B6, deposited under Budapest Treaty at the ECACC under reference number 08071704.

6. The method of claim 1 wherein the monoclonal antibody that specifically binds to free κ is produced by the hybridoma cell line B3B4, deposited under Budapest Treaty at the ECACC under reference number 08071705.

7. The method of claim 1 wherein the variable region of the heavy chain of the monoclonal antibody that specifically binds to free λ comprises the amino acid sequence provided in SEQ ID NO:5, and/or the variable region of the light chain of the monoclonal antibody that specifically binds to free λ comprises the amino acid sequence provided in SEQ ID NO:6.

8. The method of claim 7 wherein the monoclonal antibody that specifically binds to free κ is produced by the hybridoma cell line 21C3, deposited under Budapest Treaty at the ECACC under reference number 08071701.

9. The method of claim 1 wherein the monoclonal antibody, that specifically binds to free λ is produced by the hybridoma cell line 21E9, deposited under Budapest Treaty at the ECACC under reference number 08071703.

10. The method of claim 1 wherein steps (i) and (ii) are performed in the presence of labelled free κ and λ immunoglobulin light chain molecules that are capable of being bound by the monoclonal antibodies and are distinguishable from free κ and λ immunoglobulin light chain molecules in the test sample.

11. The method of claim 10 wherein steps (i) and (ii) comprises measuring the amount of the labelled κ and λ immunoglobulin light chain molecules bound to the monoclonal antibodies.

12. The method of claim 10 wherein the labelled κ and λ immunoglobulin light chain molecules are conjugated with a binding entity, preferably biotin.

13. The method of claim 10 wherein a standard curve is determined for the binding of the labelled κ and λ immunoglobulin light chain molecules bound to the assay reagent.

14. The method of claim 1 wherein the monoclonal antibody that specifically binds to free κ is attached to a first support material, the monoclonal antibody that specifically binds to free λ is attached to a second support material, the first and second support materials being distinguishable from each other.

15. The method of claim 14 wherein the support material is microspheres.

16. The method of claim 14 wherein the support material is microscopic aluminium particles.

17. The method of claim 14 wherein the support material is a slide or microplate well, where the monoclonal antibodies are separated spatially.

18. The method of claim 1 wherein steps (i) and (ii) are performed simultaneously.

19. The method of claim 1 wherein the method further comprises measuring the amount of one or more further analyte(s) in the test sample.

20. The method of claim 19 wherein the analyte(s) is albumin, whole immunoglobulins, CRP and/or B2m.

21. The method of claim 1 wherein the test sample is a mammalian tissue or biological fluid.

22. The method of claim 21 wherein the test sample is urine or, preferably, serum.

23. An isolated monoclonal antibody, or an antigen binding fragment or variant of said monoclonal antibody, that specifically binds to free κ, said monoclonal antibody comprising the amino acid sequence provided in SEQ ID NO:1 and/or SEQ ID NO:2.

24. The monoclonal antibody of claim 23 wherein the monoclonal antibody that specifically is produced by the hybridoma cell line KA1, deposited under Budapest Treaty at the ECACC under reference number 08071702.

25. An isolated monoclonal antibody, or an antigen binding fragment or variant of said monoclonal antibody, that specifically binds to free κ, said monoclonal antibody comprising the amino acid sequence provided in SEQ ID NO:3 and/or SEQ ID NO:4.

26. The monoclonal antibody of claim 25 wherein the monoclonal antibody that specifically is produced by the hybridoma cell line 1B6, deposited under Budapest Treaty at the ECACC under reference number 08071704.

27. An isolated monoclonal antibody, or an antigen binding fragment or variant of said monoclonal antibody, that specifically binds to free λ, said monoclonal antibody comprising the amino acid sequence provided in SEQ ID NO:5 and/or SEQ ID NO:6.

28. The monoclonal antibody of claim 27 wherein the monoclonal antibody that specifically is produced by the hybridoma cell line 21C3, deposited under Budapest Treaty at the ECACC under reference number 08071701.

29. An isolated monoclonal antibody, or an antigen binding fragment or variant of said monoclonal antibody, that specifically binds to free κ, said monoclonal antibody produced by hybridoma cell line B3B4, deposited under Budapest Treaty at the ECACC under reference number 08071705.

30. An isolated monoclonal antibody, or an antigen binding fragment or variant of said monoclonal antibody, that specifically binds to free λ, said monoclonal antibody produced by hybridoma cell line 21E9, deposited under Budapest Treaty at the ECACC under reference number 08071703.

31. A support material having attached a monoclonal antibody that can bind to free κ immunoglobulin light chain molecules as defined in claim 23.

32. A support material having attached a monoclonal antibody that can bind to free λ immunoglobulin light chain molecules as defined in claim 27.

33. The support material of claim 31 wherein the material is microspheres.

34. The support material of claim 31 wherein the material is microscopic aluminium particles.

35. A Hybridoma cell line selected from the group consisting of:
Hybridoma cell line KA1 with deposit reference 08071702
Hybridoma cell line 1B6 with deposit reference 08071704
Hybridoma cell line B3B4 with deposit reference 08071705
Hybridoma cell line 21C3 with deposit reference 08071701
Hybridoma cell line 21E9 with deposit reference 08071703

36. (canceled)

37. (canceled)

38. (canceled)

39. (canceled)

40. A kit for performing a method of measuring the ratio of free κ immunoglobulin light chain molecules to free λ immunoglobulin light chain molecules in a test sample comprising:
(i) a monoclonal antibody, or an antigen binding fragment or variant of said monoclonal antibody, that specifically binds to free κ; and,
(i) a monoclonal antibody, or an antigen binding fragment or variant of said monoclonal antibody, that specifically binds to free λ.

41. The kit of claim 40 wherein the monoclonal antibodies are attached to a support material as defined in claim 31.

42. The kit of claim 40 wherein the kit further comprises labelled κ and λ immunoglobulin light chain molecules that are capable of being bound by the binding molecules and are distinguishable from free κ and λ immunoglobulin light chain molecules in the test sample.

43. The kit of claim 40 wherein the kit further comprises a data sheet aiding the calculation of the ratio of free κ to free λ levels in the test sample.

44. A method of assessing the likelihood that a subject has, or may develop, a malignant plasma cell disease, an immunodeficiency, or to measure drug toxicity in subjects receiving cytotoxic therapy, comprising determining the measuring the ratio of free κ immunoglobulin light chain molecules to free λ immunoglobulin light chain molecules in a test sample from the subject according to the method of claim 1.
